(19) United States
(12) Patent Application Publication
(10) Pub. No.: US 2012/0315627 A1
(43) Pub. Date: Dec. 13, 2012

(54) METHOD FOR DETERMINING THE EFFECTIVENESS OF STERILIZATION AND/OR DISINFECTION PROCESS

(76) Inventors: Harmesh Singh Aojula, Oldham (GB); David John Clarke, Sandbach (GB); Malcolm Paul Varnham, Alresford (GB)

(21) Appl. No.: 13/561,539

(22) Filed: Jul. 30, 2012

Related U.S. Application Data
(63) Continuation-in-part of application No. PCT/GB2011/001147, filed on Jul. 29, 2011.
(60) Provisional application No. 61/512,598, filed on Jul. 28, 2011.

(30) Foreign Application Priority Data
Jul. 31, 2010 (GB) 1012875.9
Oct. 29, 2010 (GB) 1018321.8
Jul. 29, 2011 (GB) 1113212.3

(51) Int. Cl. C12Q 1/22; C12M 1/34; C12Q 1/70

(52) U.S. Cl. 435/5; 435/31; 435/288.7

(57) ABSTRACT

A method for determining the effectiveness of a sterilization and/or disinfection process is disclosed.

### CROSS REFERENCE TO RELATED APPLICATIONS

This application claims benefit under 35 U.S.C. §119(e) of provisional application U.S. Ser. No. 61/512,598, filed Jul. 28, 2011. This application also claims benefit under 35 U.S.C. §119(a)-(c) of UK Patent Application No. 1113212.3, filed Jul. 29, 2011. This application is also a continuation-in-part and claims benefit under 35 U.S.C. §119(a) and §365 of PCT Application No. PCT/GB2011/001147, filed Jul. 29, 2011; which claims priority to Great Britain Application No. 1012875.9, filed Jul. 31, 2010; and Great Britain Application No. 1018321.8, filed Oct. 29, 2010. The entire contents of each of the above-referenced patents and patent applications are hereby expressly incorporated herein by reference.

### STATEMENT REGARDING FEDERALLY SPONSORED RESEARCH OR DEVELOPMENT

Not Applicable.

### BACKGROUND OF THE INVENTION

1. Field of Invention

The invention relates to a method for determining the effectiveness of a sterilization and/or disinfection process. The presently disclosed and claimed inventive concept(s) has application in rapid sterilization procedures for autoclaves.

2. Background to the Invention

Living bacteria are referred to as vegetative cells. Vegetative cells can be killed by subjecting the cells to harsh conditions. However some bacterial cells, such as members of genera Bacillus, Clostridium and Sporosarcina, have the ability to form bacterial endospores under such conditions. Endospores are a metabolically dormant form of bacteria that can be present in high numbers when bacteria encounter inhospitable conditions, such as but not limited to, lack of nutrients. The spores can remain in this resilient state for a long time, sometimes lasting years, until favourable conditions allow germination. The structure of the spore is such that they have a core structure protected by a hardened shell of protein and carbohydrates produced by a form of binary fission in bacteria. The spore coat renders the endospores drought, heat, and starvation resistant, and also highly resistant to physical or chemical damage.

Bacillus and Clostridium endospores cause anthrax, tetanus, botulism, and gas gangrene. Endospore-forming bacteria are most commonly found in the soil, for example Desulfotomaculum, Sporolactobacillus, and Sporosarcina. Their prescence as contaminants in food can cause food spoilage.

Endospores exist almost everywhere, including the atmosphere, where they can ride on dust particles. This dormant bacterial form can survive harsh conditions such as boiling, freezing, and desiccation that readily kill vegetative bacteria. Their resilience is the primary reason for the lengthy and elaborate sterilization procedures that are employed in hospitals, canneries, and other places where sterilization is required.

Challenge organisms, such as endospores of Bacillus stearothermophilus (or Geo-bacillus stearothermophilus) are commonly used for sterilization validation studies and periodic check of sterilization cycles, such as but not limited to, in laboratory and hospital autoclaves. For example, a biological indicator containing spores of the organism on filter paper can be placed inside a vial. After sterilizing, the cap is closed, an ampoule of growth medium inside of the vial is crushed and the whole vial is incubated. A colour and/or turbidity change indicates the results of the sterilization process. No change indicates that the sterilization conditions were achieved. A colour or turbidity change indicates that the spores germinated, and the sterilization process was not successful. A disadvantage with this sterilization test is that it requires lengthy and labour intensive methods, mainly because germinating the spores can take several days.

An altogether better approach would be to detect directly whether any spores are still viable at the end of the sterilization test. Current direct methods to detect whether spores are viable include testing for dipicolinic acid (DPA).

Dipicolinic acid (DPA) is known to be present in endospores, but is not present in fungal spores, viruses, or pollen. It is a major constituent of bacterial endospores (˜10%, dry weight) and is present in all endospores. As a result, it has been used as a marker to detect bacterial spore content by providing an estimate of total spore numbers. Inside the spores it is present as a chelate, notably with calcium ions. When spores germinate, DPA and calcium ions are released while water enters the spore to allow rehydration of the core. The released DPA can be detected by a number of techniques, the most common of which involves the principle of forming a chelate with terbium and recording the luminescence of the DPA-Tb³⁺ chelate. Other approaches (Appl Environ Microbiol, p. 6808-6814 (October 2006); Journal of the American Chemical Society, 128(39):12618-12619 (2006)) have also been used to measure released DPA, including but not limited to, ultra light violet absorption, Fourier transform infra-red spectroscopy (FT-IR), fluorescence life-time measurements, iron(II)-DPA colourimetry, and Raman spectroscopy. US Patent Application Publication No. US2008/0093566 describes a method for detecting spores with ultra-violet radiation, non-destructively and without any added dyes. International Patent Application Publication No. WO03/065009 used the standard terbium assay to detect spores using an ultra violet light source and by measuring fluorescence lifetimes of the terbium. U.S. Pat. No. 5,876,960 describes the use of terbium ions to measure spores by measuring the luminescence of the terbium after chelation with DPA. In the above aforementioned methods, the spores were subjected to harsh treatments, such as heating, in order to break the coatings and release the DPA. In order to provide sufficient signal, it is necessary to release DPA from at least a million spores. This is a disadvantage because the presence of a small number of spores can be infectious. These methods are therefore not suitable for the rapid and direct detection of viable spores as required for sterilization tests.

Various fluorescent indicators are available to detect many physiologically important molecules inside cells. Examples include fluorescence probes that can bind to nucleic acids, calcium, magnesium, sodium, protons, enzymes, peptides and/or proteins. Bacterial spores can be weakly stained with dyes that bind to nucleic acids. These include DAPI and acridine orange, as well as membrane staining dyes such as di-4-ANEPPS, and a dye that binds to anionic phospholipids, 10-N-nonyl acridine orange (J Appl Microbiol., 106(3):814-824 (March 2009). Fluorescence dyes for measurement of calcium (Analytica Chimica Acta, 435:239-246 (2001)) from bacterial endospore suspensions have also been described. However, because markers such as calcium, nucleic acids and phospholipids are naturally abundant, they are not specific indicators of spore presence. Calcium in particular is not only found in most life forms, but is also frequently present (for example in hard water), making calcium a poor marker for detecting spores.

There are also probes that can be internalized into cells, where they become metabolized and yield a colorimetric or fluorescent signal. For instance, the Calcein-AM assay is used to detect esterase activity in cells as an indicator of viability. However, such viability assays that rely on metabolism or growth of cells require lengthy incubations for the metabolism to take effect before the signal becomes detectable.

The terbium DPA assay relies on an intermolecular energy transfer to cause a fluorescence change. This mechanism is not highly efficient due to non-radiative decay. Only a small part of the excited energy from DPA is transferred to the terbium ions, resulting in a relatively weak luminescence. To increase the relative luminescence (that is to improve the quantum efficiency), various other ligands have been attached to enhance the lanthanide fluorescence (Journal of Alloys and Compounds, 334(1-2):228-231 (28 Feb. 2002)). A neutral ligand like trioctyl phosphine oxide has been used as a secondary ligand to obtain a better quantum yield, but with limited success. Ligands that can shield the lanthanide complex have reduced the probability of non-radiative decay, but nevertheless the quantum yield of the complex is far from the quantum efficiencies one observes with commonly used fluorescent stains such as calcein (Halverson, et al. J. Chem. Phys., 41:157, 2752 (1964)). None of these chemical modifications are likely to solve the problems of detecting DPA in spores. Therefore while the DPA-Tb³⁺ assay (Reviewed in Analytical Chemistry, 18(1-2):1-21 (1999); and Analyst, 124(11):1599-1604 (1999)) is useful to detect the released DPA from spore suspensions, it has many shortfalls. The molar absorbtivities and quantum yields of DPA-metal chelates are poor. Such complexes do not yield as bright a fluorescence signal when compared to fluorescent dyes that are commonly used in high sensitivity assays.

Another limitation of the standard DPA-Tb³⁺ assay for detecting spores is that it generally requires the DPA to be released from spores into a solution which is then measured to record fluorescence. The DPA is diluted from a high concentration inside the spore to almost undetectable levels when suspended in large volume. Since spores are only a fraction of the size of a bacterium, it would be advantageous if it were possible to detect the DPA sequestered in a small microscopic compartment within the spore without having to first release the DPA into solution. Microscopic detection of spores, without release of DPA, would thus provide huge signal amplification over existing methods. To highlight this further, it is estimated that there can be 10⁸ DPA molecules per spore on average. The content of the DPA is relatively high inside a single spore (molar concentration in the 0.5 mM range or higher). The release of the DPA into a 1 mL volume of solution causes a signal dilution of 5×10⁹ fold. A single spore releasing its DPA content into a 1 mL solution provides a concentration of 10⁻¹³ M, which is millions of orders of magnitude below the micromolar detection limit of DPA-Tb³⁺. Thus, the terbium assay is not sensitive in practice, and requires a high number of spores to produce a detectable signal.

Another limitation of the DPA-Tb³⁺ assay is that the complex requires excitation energy by ultra-violet light, typically at 276 nm. This results in a number of problems when considering biological samples and the matrices they may be present in, since most such samples will themselves have ultra-violet absorption properties. In addition to this, microscopic applications of this assay are severely limited, as ultra-violet light sources for microscopic imaging are expensive and pose health risks to the observer.

Many fluorescent dyes are being used in fluorescence microscopy or fluorescence cytometry or other flow through devices to label objects and allow detection. In some cases the dyes are merely used as straight tags or labels. In other cases, the dye can bind to certain part(s) of the object and provide a site specific signal. In any case there can be a background signal from the non-specific binding of dye, which needs to be kept to a minimum for signal resolution. Thus, additional steps such as, but not limited to, washing, use of a flow system, and spreading the objects over larger areas are useful means to reduce background. Cytochemical fluorescent dyes as well as dyes conjugated to biomolecules (fluorescence probes) can selectively label certain cell compartments, and there are hundreds of such dyes available for microscopic and other cytometry applications (Microscopic techniques in Biotechnology, Michael Hoppert ISBN: 9783527301980). Some of the dyes with high quantum yields include Calcein and its derivatives, Fluorescein and its derivatives, teramethylrhodamine, texas red, Alexa fluor, DyLight, Cy3 and Cy5. Quantum dots and near infra red dyes are also emerging as useful labels with additional benefits. While such dyes can provide much more sensitive and highly stable signals, they have not found any use in direct detection of DPA in spores as they have do not have the ability to bind DPA specifically.

Dyes have been used to detect spores by detecting the presence of calcium, nucleic acids, and phopholipids. For example, fluorescent dyes such as calcein have been used (Analytica Chimica Acta, 435: 239-246 (2001)). However, calcium is very prevalent in biological samples and thus not a specific marker of spores. Furthermore, calcein has not been used to penetrate into spores, or detect single spores. Calcein is neither a sensitive nor a specific probe for calcium detection, as it has a large background signal in the absence of calcium, responds non-specifically to other metal ions including Al, Ba, Ca, Cu, Mg and Zn, and is reported to be sensitive to calcium in alkaline solutions only. The reason for the pH sensitivity is clear from the work of Wallach and Steck (Anal. Chem., 35:1035 (1963)) that shows that large increases in fluorescence could be measured upon complexation of calcein to calcium at pH 12, whereas only small (<12%) changes in fluorescence could be produced by complexation at neutral pH. Such studies have documented that calcein as an indicator of calcium would thus seem to be sensitive only under highly alkaline conditions. The measurement thus provides very poor contrast between fluorescence emitted from calcein bound to the calcium from a spore and background fluorescence emerging from the calcein in solution. The method is also non-specific as it is unable to detect DPA, as calcein is not able to respond to DPA.

The prior art thus provides no technique for the rapid and sensitive validation of sterilization techniques. Sensitive tests require culturing the endospores within a growth medium, a process that can take several days. Rapid tests (that do not include a culturing step) rely on releasing DPA into solution, and this greatly reduces the sensitivity of the assay. Therefore, the presently disclosed and claimed inventive concept(s) is directed to a method for determining the effectiveness of a sterilization and/or disinfection process that overcomes the disadvantages and defects of the prior art.

### BRIEF DESCRIPTION OF THE DRAWINGS

This patent or application file contains at least one drawing executed in color. Copies of this patent or patent application publication with color drawing(s) will be provided by the Office upon request and payment of the necessary fee.

FIG. 1 shows the principle of DPA detection.

FIG. 2 shows a diagram of a cross section of a bacterial spore (also known as an endospore).

FIG. 3 illustrates how DPA within a spore can be detected by the presently disclosed and claimed inventive concept(s).

FIG. 4 shows a suspension of spores in a solution.

FIG. 5 shows magnetic beads providing a local concentration of spores within the solution.

FIG. 6 shows a magnetic bead comprising an antibody.

FIG. 7 shows an optical imaging system.

FIG. 8 shows the absorption and emission of a luminescent dye.

FIG. 9 shows a graph of fluorescence intensity of a dye versus concentration of a metal ion.

FIG. 10 shows a graph of fluorescence intensity of a dye-metal complex versus concentration of DPA.

FIG. 11 shows a plot of fluorescence intensity of a dye-metal complex against increasing concentration of DPA.

FIG. 12 shows a plot of fluorescence intensity of a dye-metal complex against increasing numbers of spores.

FIG. 13 shows a plot of fluorescence intensity of terbium against type of treatment used to release the DPA from spores before its detection by adding to the terbium solution.

FIG. 14 shows a brightfield photomicrograph image of spores using a dye-metal stain.

FIG. 15 shows a fluorescence photomicrograph image of the same sample as in FIG. 14.

FIG. 16 shows a plot of fluorescence intensity of a reagent comprising a dye-metal complex encapsulated into unilamellar liposomes when released into an assay solution containing different concentrations of DPA. The arrow indicates the time point when TRITON™ X-100 is added to release the reagent.

FIG. 17 shows a fluorescence photomicrograph image of the spores using a dye-metal stain.

FIG. 18 shows a fluorescence intensity against time for the dye-metal stain when either DPA (trace A) or calcium chloride (Trace B) is added after certain (X) period of time.

FIG. 19 shows a graph of fluorescence intensity of a dye-metal versus time after the addition of spores. The dotted line is only drawn for the purpose of calculating the rate of signal formation.

FIG. 20 shows a fluorescence photomicrograph image of the spores captured with magnetic bead and stained using dye-metal stain.

FIG. 21 shows a fluorescence photomicrograph image of the Clostridium difficile spores stained using dye-metal stain.

FIG. 22 shows a bar graph of fluorescence intensity one hour after adding spores to a dye solution without the metal ions. The type of treatment used to release the DPA is given on the x-axis.

FIG. 23 shows a bar graph of fluorescence intensity one hour after adding spores to dye-metal solution. The type of treatment used to release DPA is given on the x-axis.

FIG. 24 shows a bar graph of percentage fluorescence intensity when metal is added to the dye (grey bars) and also when DPA is added (black bars) to this same metal quenched solution. The percentage fluorescence intensity was calculated by dividing the fluorescence intensity observed by the fluorescence intensity of dye alone (in the absence of the metal ions and DPA) and then multiplying by 100.

FIG. 25 shows a bar graph of percentage fluorescence intensity when metal is added to Phen Green dye (grey bars) and also when DPA is added (black bars) to this same metal quenched solution. The % fluorescence intensity was calculated by dividing the fluorescence intensity observed by the fluorescence intensity of dye alone (in the absence of the metal ions and DPA) and then multiplying by 100.

FIG. 26 shows an epifluorescence photomicrograph image of the Bacillus spores stained using dye-metal stain where the metal is europium. The scale bar is 10 microns as shown in the white box on the image.

FIG. 27 shows a fluorescence photomicrograph image of the Bacillus cereus endospores (indicated by an arrow) inside bacterial cells or sporangia when stained using dye-metal stain. The scale bar is 10 microns as shown in the white box on the image.

FIG. 28 is a graph illustrating the emission spectrum of resorufin (spectrum 1) in the presence of europium (spectrum 2) and when DPA is added (spectrum 3) to europium quenched resorufin solution.

FIG. 29 shows an epifluorescence photomicrograph image of the Bacillus cereus endospores after capturing by centrifugation and staining using dye-metal stain.

FIG. 30 shows the emission spectra of dye-metal stain, with and without DPA at the excitation wavelength of the terbium-DPA complex.

FIG. 31 shows the emission spectrum of 0.12 μM solution of a Calcein-terbium stain (no chelator spectrum) using an excitation wavelength of 485 nm with and without the 100 μM of chelator addition.

FIG. 32 shows similar results to FIG. 31 for chelators that showed a much smaller change in fluorescence compared to the chelators of FIG. 31.

FIG. 33 shows a method for determining the effectiveness of a sterilization and/or disinfection process according to the presently disclosed and claimed inventive concept(s).

FIG. 34 shows an autoclave produced in accordance with the presently disclosed and claimed inventive concept(s).

FIG. 35 shows that the detection of dye encapsulated inside a compartment at self quenching concentration can be followed by relief of self quenching as it becomes diluted when released from the compartment.

FIG. 36 shows the fluorescence emission spectra of samples of luminescent endospores before (spectrum 1) and after (spectrum 3) autoclaving. The figure also shows spectrum 2 when the dye-metal alone is autoclaved.

FIG. 37 shows photomicrograph images of luminescent endospores before (panel A) and after (Panel B) 15 minutes of autoclaving, wherein the stain was added after the autoclaving.

FIG. 38 show photomicrograph images of luminescent endospores before (panel A) and after (panel B) 15 minutes of autoclaving, wherein the stain was added before the autoclaving.

### DETAILED DESCRIPTION OF THE INVENTIVE CONCEPT(S)

Before explaining at least one embodiment of the inventive concept(s) in detail by way of exemplary drawings, experimentation, results, and laboratory procedures, it is to be understood that the inventive concept(s) is not limited in its application to the details of construction and the arrangement of the components set forth in the following description or illustrated in the drawings, experimentation and/or results. The inventive concept(s) is capable of other embodiments or of being practiced or carried out in various ways. As such, the language used herein is intended to be given the broadest possible scope and meaning; and the embodiments are meant to be exemplary—not exhaustive. Also, it is to be understood that the phraseology and terminology employed herein is for the purpose of description and should not be regarded as limiting.

Unless otherwise defined herein, scientific and technical terms used in connection with the presently disclosed and claimed inventive concept(s) shall have the meanings that are commonly understood by those of ordinary skill in the art. Further, unless otherwise required by context, singular terms shall include pluralities and plural terms shall include the singular. Generally, nomenclatures utilized in connection with, and techniques of, cell and tissue culture, molecular biology, and protein and oligo- or polynucleotide chemistry and hybridization described herein are those well known and commonly used in the art. Standard techniques are used for recombinant DNA, oligonucleotide synthesis, and tissue culture and transformation (e.g., electroporation, lipofection). Enzymatic reactions and purification techniques are performed according to manufacturer's specifications or as commonly accomplished in the art or as described herein. The foregoing techniques and procedures are generally performed according to conventional methods well known in the art and as described in various general and more specific references that are cited and discussed throughout the present specification. See e.g., Sambrook et al. Molecular Cloning: A Laboratory Manual (2nd ed., Cold Spring Harbor Laboratory Press, Cold Spring Harbor, N.Y. (1989) and Coligan et al. Current Protocols in Immunology (Current Protocols, Wiley Interscience (1994)), which are incorporated herein by reference. The nomenclatures utilized in connection with, and the laboratory procedures and techniques of, analytical chemistry, synthetic organic chemistry, and medicinal and pharmaceutical chemistry described herein are those well known and commonly used in the art. Standard techniques are used for chemical syntheses, chemical analyses, pharmaceutical preparation, formulation, and delivery, and treatment of patients.

All patents, published patent applications, and non-patent publications mentioned in the specification are indicative of the level of skill of those skilled in the art to which this presently disclosed and claimed inventive concept(s) pertains. All patents, published patent applications, and non-patent publications referenced in any portion of this application are herein expressly incorporated by reference in their entirety to the same extent as if each individual patent or publication was specifically and individually indicated to be incorporated by reference.

All of the compositions and/or methods disclosed and claimed herein can be made and executed without undue experimentation in light of the present disclosure. While the compositions and methods of the inventive concept(s) have been described in terms of preferred embodiments, it will be apparent to those of skill in the art that variations may be applied to the compositions and/or methods and in the steps or in the sequence of steps of the method described herein without departing from the concept, spirit and scope of the presently disclosed and claimed inventive concept(s). All such similar substitutes and modifications apparent to those skilled in the art are deemed to be within the spirit, scope and concept of the inventive concept(s) as defined by the appended claims.

As utilized in accordance with the present disclosure, the following terms, unless otherwise indicated, shall be understood to have the following meanings:

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects. For example but not by way of limitation, when the term “about” is utilized, the designated value may vary by plus or minus twelve percent, or eleven percent, or ten percent, or nine percent, or eight percent, or seven percent, or six percent, or five percent, or four percent, or three percent, or two percent, or one percent. The use of the term “at least one” will be understood to include one as well as any quantity more than one, including but not limited to, 2, 3, 4, 5, 10, 15, 20, 30, 40, 50, 100, etc. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y and Z” will be understood to include X alone, Y alone, and Z alone, as well as any combination of X, Y and Z. The use of ordinal number terminology (i.e., “first”, “second”, “third”, “fourth”, etc.) is solely for the purpose of differentiating between two or more items and is not meant to imply any sequence or order or importance to one item over another or any order of addition, for example.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

The term “disinfection process” as used herein will be understood to include any method of disinfection for which the method described and claimed herein below may be utilized to determine the effectiveness of said disinfection method. The term “disinfection process” will further be understood to include disinfection, antiseptic and/or antimicrobial treatments.

Turning now to the presently disclosed and claimed inventive concept(s), there is provided a method for determining the effectiveness of a sterilization and/or disinfection process. In one embodiment of the method, a biological indicator comprising at least one live organism into which a luminescent material has been introduced is exposed to a sterilization and/or disinfection process, and the biological indicator is excited with an excitation energy distinctive of the luminescent material. In another embodiment, the at least one live organism is exposed to the sterilization and/or disinfection process, and the luminescent material is introduced therein following exposure to the sterilization and/or disinfection process. In either embodiment, the luminescence of the biological indicator is then measured, and the effectiveness of the sterilization and/or disinfection process is determined based on the luminescence measured. The live organism comprises a permeability layer that retains the luminescent material, whereby the killing of the organism by the sterilization and/or disinfection process permits the luminescent material to pass through the permeability layer of the dead organism, thereby changing the luminescence of the biological indicator. In certain embodiments, the determination step may be based on an initial known number of the live organisms in said biological indicator.

Any live organism known in the art or otherwise contemplated herein that is capable of functioning in accordance with the presently disclosed and claimed inventive concept(s) may be utilized. For example, the live organism may be (for example but not by way of limitation) a fungal spore, a protozoan spore or cyst, a dehydrated animal or protozoan cell, a virus, a fungal mycelium, and/or a bacterial cell. In certain embodiments of the presently disclosed and claimed inventive concept(s), the live organism may be in a viable and dehydrated state. In another embodiment, the live organism may be a bacterial endospore, and the permeability layer may be a spore coat. In this embodiment, the endospore may be a viable, non-germinating spore. In a particular embodiment, the bacterial endospore may also comprise dipicolinic acid and/or a derivative thereof.

Any luminescent material known in the art or otherwise contemplated herein that is capable of functioning in accordance with the presently disclosed and claimed inventive concept(s) may be utilized. In certain embodiments, the luminescent material may be a luminescent dye. The luminescent dye may have been introduced into the endospore by binding it to a metal ion to form a dye-metal complex and contacting the endospore with the dye-metal complex. The luminescent dye may be quenched by the metal ion, and the dye-metal complex may become unquenched when the luminescent dye is released from the dye-metal complex. Alternatively, the luminescent dye may be such that it self-quenches above a certain concentration, and the concentration of the luminescent dye in the compartment may be such that the luminescent dye is self quenched prior to the sterilization and/or disinfection process; in this manner, the luminescent dye may become unquenched as a result of the sterilization and/or disinfection process. In either of these embodiments, the unquenching may occur in response to the dye-metal complex contacting DPA or derivative thereof. Alternatively, the unquenching may occur in response to dilution of the dye from the endopsore into the surroundings. The luminescent dye may be such that it is not quenched by calcium. In addition, the method may include the step of adding a quenching chemical, such as but not limited to, cobalt ions, to reduce the background and non-specific signal arising from free dye.

In certain embodiments, the luminescent dye may be a fluorescent dye. Any fluorescent dye known in the art or otherwise contemplated herein that is capable of functioning in accordance with the presently disclosed and claimed inventive concept(s) may be utilized. Examples of fluorescent dyes that may be used in accordance with the presently disclosed and claimed inventive concept(s) include, but are not limited to, Calcein, Fluorescein, rhodamine, texas red, Alexa fluor, DyLight, Cy3 and Cy5, Quantum dots and near infra red dyes, fura dyes, resorufin, derivates thereof, and combinations thereof.

Any metal ion known in the art or otherwise contemplated herein that is capable of functioning in accordance with the presently disclosed and claimed inventive concept(s) may be utilized. Non-limiting examples of metal ions include, but are not limited to, a rare earth metal ion (such as but not limited to, lanthanum, cerium, praseodymium, neodymium, promethium, samarium, europium, gadolinium, terbium, dysprosium, holmium, erbium, thulium, ytterbium, and/or lutetium), cobalt, copper, nickel, zinc, manganese, iron, lead, cadmium, mercury, and combinations thereof. The metal ion may be selected such that when it binds to the first chelator (for example DPA), it forms a first chelate, which chelate has a higher luminescence than the metal ion. When the chelate binds to the luminescent dye, it forms a second chelate, and this second chelate has lower (quenched) luminescence when compared to the luminescent dye. The luminescence of the dye in a viable spore may be greater than the luminescence of the dye in a non-viable spore, or the luminescence of the dye in a viable spore may be lower than the luminescence of the dye in a non-viable spore.

The presently disclosed and claimed inventive concept(s) extends to an autoclave in which a sterilization procedure is performed, wherein said sterilization procedure has been confirmed using any method described or otherwise contemplated herein.

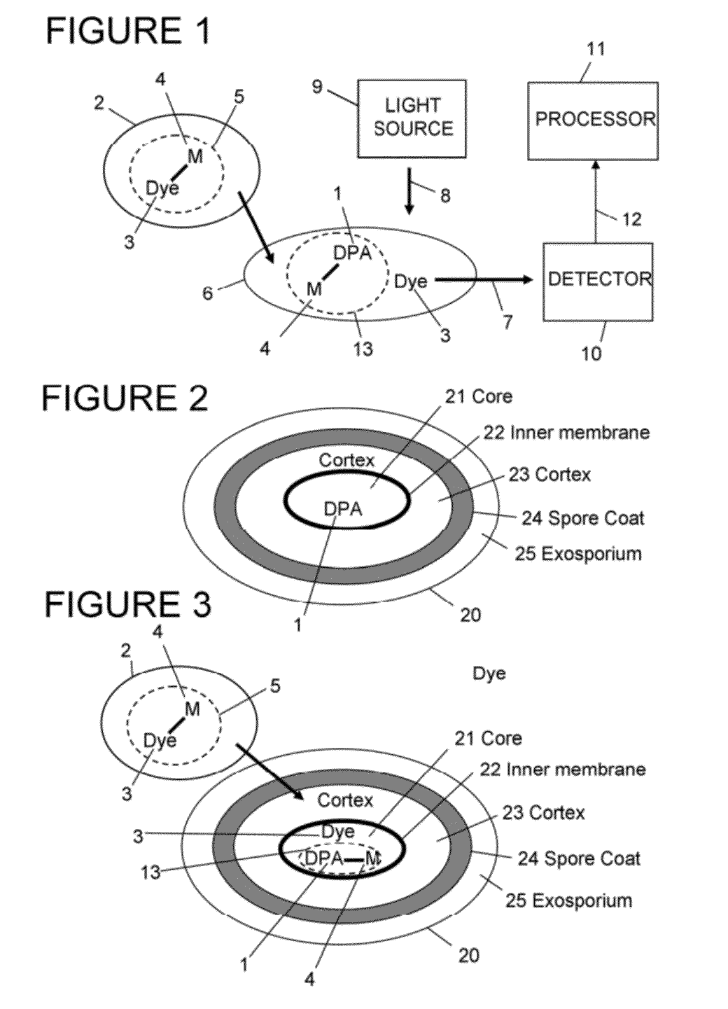

Turning now to the Figures, FIG. 1 shows a method for detecting a first chelator (shown in the figure as dipicolinic acid DPA 1), the method comprising the steps of providing a reagent 2 comprising a second chelator (shown as a luminescent dye 3) and a metal ion 4, contacting the reagent 2 with a sample 6 containing the DPA 1, exciting a luminescence 7 of the dye 3, and detecting the luminescence 7 emitted by the dye 3, the method being characterized in that the metal ion 4 is bound to the luminescent dye 3 within the reagent 2, the luminescence 7 of the dye 3 is altered by the metal ion 4, the metal ion 4 is capable of binding to the DPA 1, and the dye 3 and the metal ion 4 are such that the DPA 1 can compete for the metal ion 4 in competition with the dye 3, thereby re-establishing the luminescence 7 of the dye 3.

The alteration of the luminescence 7 can be in a positive or a negative sense. Thus the luminescent dye 3 can be such that the luminescence 7 is quenched by the metal ion 4, and dequenched in the presence of the DPA 1. For instance this occurs when the luminescent dye 3 is calcein, and the metal ion 4 is terbium. Alternatively, the luminescent dye 3 can be such that the luminescence 7 is enhanced by the metal ion 4, and re-established when the metal ion 4 is chelated in the presence of the DPA 1. An example is where the dye 3 is a charge transfer exciplex comprising pyrene, and the metal ion 4 is silver.

Also shown in FIG. 1 is a light source 9 selected to provide pump radiation 8 for exciting the luminescent dye 3, a detector 10 for detecting the luminescence 7, and a processor 11 for processing the output 12 of the detector 10. The light source 9 can be such that it only excites the luminescent dye 3. Alternatively, the light source 9 can be selected to excite both the luminescent dye 3 and a luminescence of the metal ion 4. This latter approach can provide additional signal amplification and enhanced selectivity.

The luminescent dye 3 and the metal ion 4 are shown as forming a dye metal-ion complex 5 within the reagent 2. When this complex is contacted with the DPA 1, the metal ion 4 is displaced by the DPA 1, and the luminescence 7 of the dye 3 becomes dequenched (that is, relieved or in other words increased in intensity). The dye metal-ion complex 5 forms an equilibrium with the dye 3 and the metal ion 4 that is controlled by the DPA 1, as shown in the following equilibrium equation.

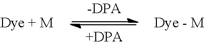

On the right hand side, the dye 3 and the metal ion 4 are shown complexed, that is, they are bound together. Adding the DPA 1 (as shown by the bottom arrow) shifts the equilibrium to the left, where the dye 3 and the metal ion 4 are shown in free form, that is, unbound. Similarly, removing the DPA 1 (as shown by the top arrow) shifts the equilibrium to the right, and the dye 3 and the metal ion 4 become bound. The fraction of the dye 3 in the bound and the unbound state is determined by the level of the DPA 1 present. The intensity of the luminescent signal 7 resulting from the unbound dye 3 (on the left hand side) is indicative or proportional to the concentration of the DPA 1 present. To be clear the luminescence of dye in the bound state is quenched and that in the unbound state is dequenched. By “quench” it is meant that the luminescent intensity becomes low and by “dequench” it is meant that the luminescent intensity increases from the quenched state, and preferably is re-established to its pre-quenched state.

Referring to FIG. 1 again, the arrow extending from the reagent 2 to the sample 6 indicates adding the reagent 2 to the sample 6. Once added, the reagent 2 encounters the DPA 1, the metal ion 4 transfers from the dye 3 to the DPA 1, thus forming the DPA metal complex 13. The dye 3 is then unbound and is no longer quenched.

The presently disclosed and claimed inventive concept(s) is particularly advantageous for detecting particles, such as bacterial endospores (or spores), that contain or release DPA. FIG. 2 shows a cross-section of a spore 20. The spore 20 comprises a core 21, and a cortex 23 that surrounds the core 21. The cortex 23 is enveloped by a spore coat 24 (shown shaded grey), which in certain spores is surrounded by an exosporium 25. The core 21 and the cortex 23 are separated by an inner membrane 22. For clarification purposes, the label 21 points to an arbitrary region within the core 21, the label 23 points to an arbitrary region within the cortex 23, and the label 25 points to an arbitrary region within the exosporium 25. DPA 1 is shown located within the core 21 of the spore 20; it is typically present as calcium dipicolinate. Depending on the nature and the state of the spore, DPA may also be present in the other compartments. As used herein, DPA is meant to include not only dipicolinic acid, but also a compound of the DPA as defined above. The core 21 also comprises deoxyribonucleic acid (DNA). The core 21, cortex 23, and the exosporium 25 are also referred to as “compartments”.

The presently disclosed and claimed inventive concept(s) described with reference to FIG. 1 is an example of a more general invention for detecting a chelator such as the DPA 1. Suitable chelators are those that have high binding affinity to lanthanides. Such chelates include polyamino carboxylic acids such as ethylenediaminetetraacetic acid (EDTA), dipicolinic acid, diaminopimelic acid (DAP), n-acetlymuramic acid, sulfolactic acid, and phosphoglyceric acid. The chelators may be ethylenediaminetetraacetic acid (EDTA). The chelator may be a related marker that is found within spores, such as dipicolinic acid DPA, diaminopimelic acid (DAP), n-acetlymuramic acid, sulfolactic acid, or phosphoglyceric acid. The chelator may in the form of a chelate within the spore.

Endospores are probably the most durable form of life known. Mature spores have no detectable metabolism, a state that is described as cryptobiotic. They can remain viable for extremely long periods of time, perhaps millions of years. They are highly resistant to environmental stresses such as high temperature (some endospores can be boiled for several hours and retain their viability), irradiation, strong acids, disinfectants, etc. Although cryptobiotic, they retain viability indefinitely such that under appropriate environmental conditions, they germinate into vegetative cells. Their durability is assisted by the spore coat 24, which in many spores comprises calcium. The exosporium 25 and the spore coat 24 also makes the spores 20 highly resistant to simple dyes and stains.

Despite their renowned durability, the inventors have discovered that it is possible to penetrate the exosporium 25 and the spore coat 24 with a luminescent dye 3 that has been quenched with a metal ion 4. The principle is shown in FIG. 3. As in FIG. 1, the reagent 2 comprises the luminescent dye 3 and the metal ion 4. They form the complex 5. Upon penetrating the spore coat 24, the luminescence of the dye 3 is dequenched when it encounters DPA either in the core 21 or the cortex 23, and the dye 3 can be detected, for example according to the method described with reference to FIG. 1. Without limiting the scope of the presently disclosed and claimed inventive concept(s) in any way, it is believed that upon penetrating the spore coat 24, the DPA 1 displaces the dye 3 from the metal ion 4, thus relieving the luminescence of the dye 3. The luminescence of the dye 3 is thus indicative of the presence of the DPA 1 within the spore 20. The intensity of the luminescence 7 is indicative or proportional to the amount of the DPA 1 present. The DPA 1 and the metal ion 4 are shown as forming the DPA-metal complex 13 within the core 23. However a similar complex can form in any compartment where the DPA 1 may be present.

The DPA 1 may be present as DPA or as calcium dipicolinate within the spore 20, for example within the spore core 21, spore cortex 23, spore coat 24 or the exosporium 25 depending on the state of the spore. This is because when the spore forms, DPA will leach into the outer layers of the spore, including the mother cell. As the mother cell dies away, any DPA 1 present will leach away. Similarly, if the spore 20 becomes damaged (for example by cleaning with sporocidal agents, including physical treatments such as heat and sonication, the DPA leaks out, including into the outer spore layers and into the bulk solution. If the spore at that point does not germinate, then it is easily damaged and dies. Consequently, the amount of DPA present and its location is an indicator of the viability of the spore. The state of the spore may be viable, non-viable, viable non-culturable, or damaged. Therefore depending on the state of the spore the DPA 1 may leach from one compartment to another. Hence it may be possible to determine the state of the spore 1 depending on which compartments become stained or on the relative intensities of staining within the different compartments.

This discovery provides significant advantages over the prior art. It was previously known to detect spores 20 by using dyes that detect the presence of calcium, nucleic acids and phopholipids. Without limiting the presently disclosed and claimed inventive concept(s) in any way, it is believed that the poor penetration may be because dyes used to stain calcium, DNA and phospholipids are highly charged and therefore are only able to weakly penetrate the spore 1 resulting in poor staining.

The presently disclosed and claimed inventive concept(s) of using the luminescent dye 3 that has been quenched with the metal ion 4, to penetrate the spore 20 solves the poor contrast problems of simply detecting calcium, as well as the weak signal problems of using a luminescent metal ion. Importantly, it also allows, a single compartment, such as the cortex 23 or the core 21, which is rich in DPA, to be preferentially stained by the dye 3. The presently disclosed and claimed inventive concept(s) allows stained spore compartment to be imaged with an imaging system such a microscope. The ability to preferentially stain a DPA rich compartment within the spore is believed to have important implications for researching and exploiting the microbiology of spores.

The dye-metal complex 5 is able to permeate into the spore 20. If the spore 20 comprises an exosporium 25, the dye-metal complex 5 is able to cross the exosporium 25. Many spores 20 contain DPA, for example within the core 21 or the cortex 23. Thus exciting the luminescence 7 of the dye 3 by the method described with reference to FIG. 1 allows the spore 20 to be detected, for example, by imaging with an optical microscope. It also allows many of the spores 20 to be detected within the sample 6.

Alternatively or additionally, the DPA 1 can be released from the spore 20 into solution. This can be achieved, for example, by physical (heat, microwave, sonication), chemical (dodecylamine) or biochemical (germinating spores with L-alanine). The DPA 1 can then be detected by the method described with reference to FIG. 1.

The presently disclosed and claimed inventive concept(s) therefore provides a method for detecting at least one spore 20 containing the DPA 1, which method includes the steps of providing the quenched luminescent dye-metal complex 5, which complex 5 comprises the luminescent dye 3 and the metal ion 4, releasing the DPA 1 from the at least one spore 20 into a solution (not shown). The quenched luminescent dye metal complex 5 can then be contacted with the DPA 1, for example, by adding the complex 5 to the solution. The luminescent dye 3 can then be excited with the light source 9 to emit the luminescent signal 7, and luminescent signal 7 detected with a detector 10. It is believed that the luminescence of the quenched luminescent dye metal-complex 5 becomes dequenched when in contact with the DPA 1.

Alternatively or additionally, the method can be used to detect at least one spore 20 which contains a spore coat 24, which method includes the steps of providing the quenched luminescent dye-metal complex 5, which complex 5 comprises the luminescent dye 3 and a metal ion 4 and causing the dye-metal complex 5 to cross the exosporium 25, for example by adding the complex 5 to the sample 6. The luminescent dye 3 can then be excited with a light source 9 to emit a luminescent signal 7, which can be detected with a detector 10.

As will be shown below, the dye-metal complex 5 can be selected to target a specific type of spore. For example, viable or mature bacterial endospores contain DPA, whereas non-viable or immature bacterial endospores either contain no DPA, or much less DPA. Thus spores in different stages of maturation and different viable states can have varying levels of DPA. It is known that when spores germinate, the spore coat and the inner membranes become more permeable, such that high levels of DPA is released from the core through the core cell inner membrane through the cortex over a period of hours. During this time the germinating spore can be stained, and the stain would penetrate more deeply into the spore, including into the core. After a period of hours, once all or some of the DPA has leached away, the spores do not stain as brightly as prior to leaching, and may not stain at all. Viable spores will have more intense luminescent signals than non-viable spores. The germinating spores will have luminescent signals that will extend further into the spore and depending on the extent of germination, into the core. Germinating spores will have the lowest strength luminescent signal. Analyzing the luminescent signal 7, for example, by comparing the strength of the luminescent signal 7 with the strength from previously known samples, or by mapping the relative concentrations of the luminescent signal within the spore thus provides a method for the detection of intact viable and non-viable spores, as well as germinating or germinated spores.

Similarly, pollen, viruses, and spores from fungi do not contain DPA. Thus the presently disclosed and claimed inventive concept(s) can be used to distinguish spores that contain DPA (such as bacterial endospores) from pollen, viruses, and spores that do not contain DPA (such as fungal spores) by using a luminescent dye metal complex 5 that is dequenched relieved by the DPA 1 present in the spore 20.

Preferably, the metal ion 4 and the dye 3 should be selected such that the metal ion 4 quenches the dye 3 more strongly than calcium ions on a molar basis. This ensures that the calcium in the spores or the sample will not have significant effect on diminishing the fluorescence of dye 3 thus allowing higher sensitivity. To provide further signal amplification, the dye 3 may be such that it has a higher fluorescence in presence of calcium once the dye 3 has been dequenched by DPA.

The luminescent dye 3 can be a fluorescent dye that is quenched by the metal ion 4 but not by calcium. Alternatively or additionally, the fluorescent dye can be selected from the list provided in Table 1. These have different excitation and fluorescent wavelengths, some of which may be more convenient than others for different applications.

TABLE 1

Fura-2, Indo-1 and derivatives
Quin-2 and derivatives
Fura-4F, Fura-5F & Fura-6F; Benzothiaza-1 & Benzothiaza-2
Fura-FF, BTC, Mag-Fura-2, Mag-Fura-5, Mag-Indo-1
Fluo-3, Rhod-2 and related derivatives
Fluo-5N, Rhod-5N, X-Rhod-5N and related derivatives
Calcium Green, Calcium Orange and Calcium Crimson indicators
Oregon Green 488
Fura Red indicator
Calcein and its derivates
Resorufin and its derivatives

Preferably, the metal ion 4 has a higher affinity for the DPA 1 than calcium. The DPA 1 can then efficiently displace the metal ion 4 even when the DPA 1 is present as calcium dipicolinate.

The metal ion 4 can be monovalent or multivalent. The metal ion 4 can be a rare earth ion. The rare earth ion can be a lanthanide, which may be selected from the group consisting of lanthanum, cerium, praseodymium, neodymium, promethium, samarium, europium, gadolinium, terbium, dysprosium, holmium, erbium, thulium, ytterbium, and lutetium. In an embodiment, the rare earth ion is europium or terbium.

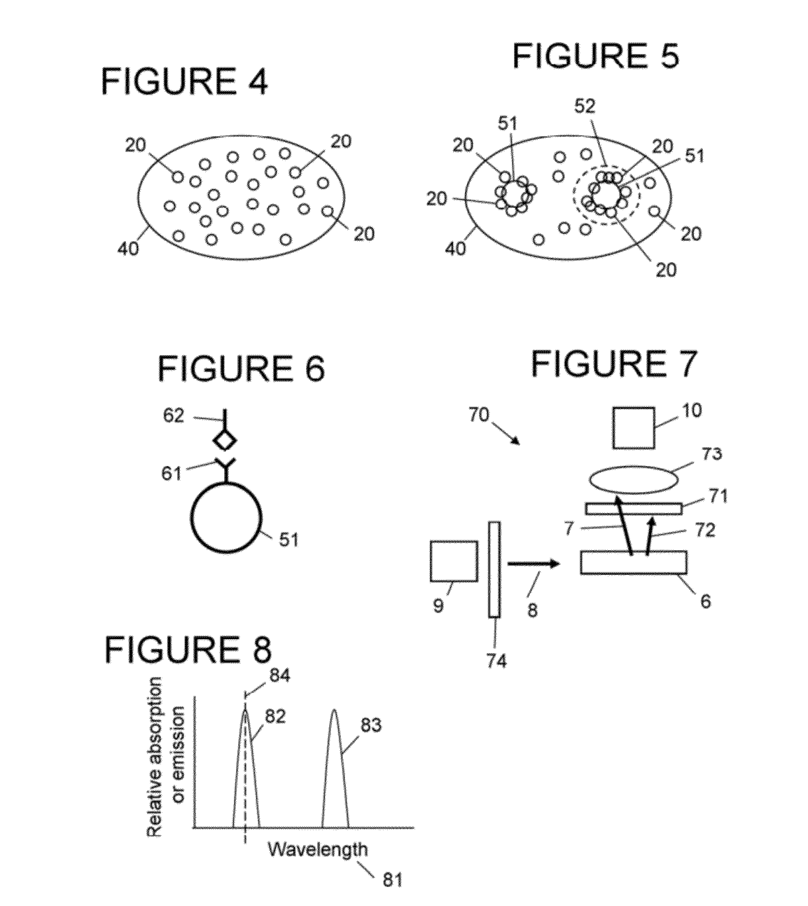

The sample 6 can comprise a plurality of the spores 20 in a solution 40, as shown with reference to FIG. 4. As shown in FIG. 5, the above methods may include the step of concentrating a local concentration 52 of the spores 20 in the solution 40. The concentration step can be performed using at least one bead 51. As shown in FIG. 6, the bead 51 may comprise an antibody 61 capable of binding to an antigen 62 that is characteristic of the spore 20. The bead 51 may be any solid particle. For instance it may be a polymer such as latex, a metal such as a paramagnetic or magnetic bead, a particle such as a liposome, or simply a solid surface. Alternatively or additionally, the concentration step may be performed using a centrifuge or a flow cytometer, filtration, adsorption and absorption onto surfaces or particles, aerosol or air sampler, Alternatively or additionally, the released DPA may be concentrated or captured by chromatography or an immunostrip before detection by the dye-metal complex 5. The antibody 61 either alone or in combination with the bead 51 can also be used to detect the presence of a single spore 20. Whether for single spores or a plurality of spores, use of the antibodies can increase the specificity of an assay.

FIG. 7 shows how an optical imaging system 70 may be used with the above methods. The sample 6 is illuminated by the pump radiation 8 from the optical source 9. The luminescence 7 of the dye 3 is shown being imaged onto the detector 10 by the lens 73. FIG. 8 shows a graph of the absorption band 82 and the emission band 83 of the luminescent dye 3 versus wavelength 81. The wavelength 84 of the pump radiation 8 can be within the absorption band 82. Alternatively, the wavelength 84 can be a multiple (such as two times or three times) the wavelength of the absorption band if the excitation of the luminescent dye 3 is by multi-photon absorption, which has advantages in terms of localization, avoiding unwanted overlap of excitation and emission wavelengths, and the ability to use cheaper plastics and other materials in the system that will not auto fluoresce at these longer excitation wavelengths. An excitation filter 74 can be provided to remove any unwanted pump radiation from the source 9 reaching the sample 6. The unwanted radiation comprises radiation wavelengths that are not able to excite the dye 3. Referring again to FIG. 7, a filter 71 can be provided to remove any unwanted signals 72 emerging from the sample 6. The unwanted signal 72 can be scattering of the pump radiation 8, or can be an unwanted luminescence of plastics or other materials used to hold the sample 6. The filters 71 and 74 can comprise at least one of an optical filter such as an optical interference filter, a grating, a monochromator, or a double monochromator. The detector 10 can comprise a semiconductor detector, a photomultiplier, a charge coupled device (CCD) array, a camera, or can be provided by the human eye.

If the detector 10 is sufficiently close (preferably less than 1 mm) to the sample 6, then the lens 73 can be emitted, and the optical imaging system 70 is then a lensless optical imaging system. If the detector 10 is a semiconductor array, such as a CCD array or a CMOS array, then the optical imaging system can provide lensless imaging onto the array. Such an array can be used to provide images of spores contained within the sample 6, such imaging being provided by refraction, diffraction, or a combination of refraction and diffraction. Then the lensless optical imaging system would include the filter 71, preferably in the form of an optical coating. The lens 73 can also be a fiber optic bundle for transmitting the signal 7 to the detector 10 which is preferably in the form of a CCD array or a CMOS array. The fiber optic bundle can provide magnification. Fiber optic bundles are available from Roper Scientific GmbH of Ottobrunn, Germany.

The above methods can thus provide a method for detecting at least one spore 20, which method includes the steps of inserting the luminescent dye-metal complex 5 into the at least one spore 20, and detecting the signal 7 from the inside of the spore 20.

The methods described with reference to FIGS. 1 to 8 provide a method for detecting at least one spore 20, which method includes the steps of causing a luminescent dye 3 and a metal ion 4 to penetrate into the at least one spore 20, and detecting the luminescence 7 of the dye 3 using an optical imaging system 70. Advantageously, these methods can include the use of the antibody 61, either alone, or in combination with a surface such as the bead 51, to provide a specific assay for a spore.

The above methods are particularly advantageous when the at least one spore 20 is a bacterial endospore.

Referring again to FIG. 3, the dye 3 is bound to the metal ion 4 to form a dye-metal complex 5 that quenches the luminescence 7 of the dye. When this dye metal complex 5 encounters DPA 1, for example the DPA 1 present inside the spore 20, the metal ion 4 becomes dissociated from the dye 3 resulting in dequenching or relieving of the dye luminescence 7 thereby increasing its luminescence. If the dye 3 is retained near the spore 20 or in the compartment such as the core 21 or the cortex 23, the dye 3 acts as a fluorescent stain providing a localized higher-intensity signal. If the dye 3 is released outside the spore 20 or a bacteria, the strength of the luminescence 7 emitted can provide a measure of the amount of the DPA 1 present, and hence can provide the total spore count. Alternatively or additionally, the stained spores 20 may be counted microscopically to determine the total count. Such a counting technique can be used to provide reference or calibration data in order to provide the total viable spore count from the strength of the luminescence 7 and the number of spores.

Suitable metal ions 4 are those which quench the luminescence 7 of the dye 3. To find a suitable metal ion 4, the dye 3 is dissolved in buffer and its luminescence emission 7 recorded. Then the test metal ions 4 are added and the change in luminescence emission recorded. Preferably the suitable metal ion 4 will result in lowering of the luminescence signal 7. Once a suitable ion is identified it further needs to be confirmed that the metal ion 4 is also capable of binding to the DPA 1 (or to another suitable target) in the presence of the dye 3. This can be done in number of ways, for example, by direct binding assays with metal using spectroscopy or any of the other methods which are commonly used to measure equilibrium constants. Preferably the DPA 1 is added to the metal quenched dye 5, and dequenching observed to indicate displacement of the metal 4 from the dye 3. When both these criteria are met final verification can be made by preparing a mixture of the metal dye complex 5 having a quenched signal and adding the DPA 1 to this solution. The luminescence emission 7 will increase when the DPA 1 is added if the test has been successful. A third criteria may need to be fulfilled when testing the DPA 1 from spores 20. In this case the metal quenched dye 5 must be able to show recovery of luminescent signal 7 when the DPA 1 is added in the presence of calcium. This is because calcium is usually found in the spores 20 associated with DPA 1 or other chemical chelators. When the metal dye complex 5 is used as a stain a fourth criteria may need to be fulfilled. In this case the metal quenched dye 5 must be able to penetrate into the spore to contact DPA within one or more of its compartments.

Lanthanide ions may be preferred as the metal ion 4, as they are known to have high affinity for DPA 1. However, in addition the lanthanide ions need to be able to quench the luminescence 7 of the dye 3.

Alternatively or additionally, to find a suitable dye 3, the metal ion 4 may be screened first with its ability to bind to the chemical chelators such as DPA 1, and then this metal ion 4 may be tested with respect to its ability to change and preferably quench the luminescence 7 of the dye 3.

There are many other techniques to study binding of metal ions 4 to chelators and dyes including dialysis, chromatography, spectroscopy. Any such technique may be used to identify a suitable metal ion 4 and dye 3. The key aspect is that the metal ion 4 must be able to compete for binding to DPA and the dye. Ideally the competition is in favor of DPA binding to the metal ion 4 rather than the dye binding to the metal ion 4. This can be assured by selecting a metal ion 4 that has a higher affinity to DPA than the dye.

Bacterial spores are often named as endospores because they are formed intracellular, although they are eventually released from their mother cell as free spores. The term “spore” includes endospore and can be used interchangeably when discussing DPA diagnostic applications. Luminescence means emission of photons from the dye 3 after it has been excited at its excitation wavelength. Fluorescence is a form of luminescence.

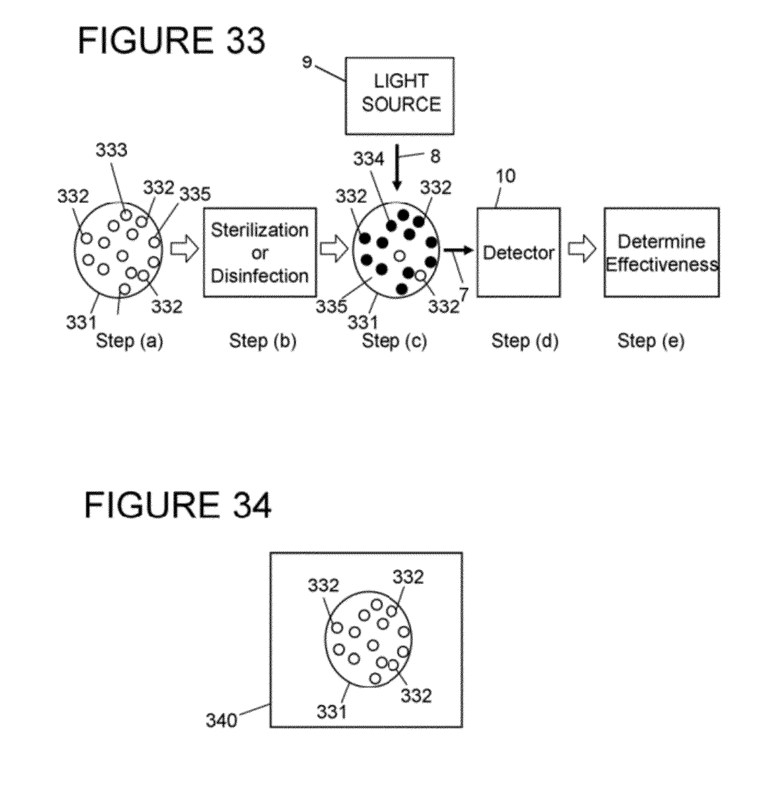

FIG. 33 shows a particular embodiment of the presently disclosed and claimed inventive concept(s), in which a method for determining the effectiveness of a sterilization and/or disinfection process includes the following steps: (a) providing a biological indicator 331 comprising at least one live organism 332 into which a luminescent material 333 has been introduced; (b) exposing said biological indicator 331 to the sterilization and/or disinfection process; (c) exciting the organism 332 with an excitation energy 8 distinctive of the luminescent material 333; (d) measuring the luminescence 7 of the biological indicator 331; (e) determining the effectiveness of the sterilization and/or disinfection process based on step (d) wherein the live organism 332 comprises a permeability layer 335 that retains the luminescent material 333; and killing the organism 332 by sterilization and/or permits the luminescent material 333 to pass through the permeability layer 335, thereby changing the luminescence 7 of the biological indicator 331.

Also shown in FIG. 33 is a light source 9 for providing the excitation energy (shown as optical pump radiation 8), and a detector 10 for detecting the luminescence 7. Organisms 332 that have been killed are indicated by the dark spots 334.

The live organism 331 may be in a viable and dehydrated state.

The determination step may be based on an initial known number of the live organisms 331 in said biological indicator 330. For example, at least 100 organisms would be required to demonstrate a 99% kill, more than 1000 organisms for a 99.9% kill, and more than 10000 organisms for a 99.99% kill. It may be necessary to multiply these number of organisms in each case by a factor of 2 to 1000 depending upon the statistical level of certainty required in the sterilization and/or procedure.

The presently disclosed and claimed inventive concept(s) is particularly useful in determining the effectiveness of a sterilization and/or disinfection process in killing bacterial endospores, for example in an autoclave. This is because spores are one of the most resistant forms of life known, and if the process kills the spores, then it is highly probable that other forms of life have also been killed. In this case, the live organism 331 is a bacterial endospore 20, and the permeability layer is a spore coat 24, which is shown in FIG. 2.

### EXAMPLES

Examples are provided hereinbelow. However, the presently disclosed and claimed inventive concept(s) is to be understood to not be limited in its application to the specific experimentation, results and laboratory procedures. Rather, the Examples are simply provided as one of various embodiments and are meant to be exemplary, not exhaustive.

### Example 1

Several fluorescence dyes, including fluorescein, rhodamine and calcein, were screened for their ability to bind to zinc, cobalt, terbium, aluminum, and several other metal ions. Their fluorescence emission was recorded before and after adding the metal ion. Many of these show quenching of the fluorescence. One of the efficient dyes was calcein whose fluorescence was quenched by a number of different metal ions including ions of zinc, cobalt, iron and terbium. These ions were tested for their ability to bind to DPA. The binding ability to DPA is variable in terms of affinity. Preferably the metal ion and the dye are selected such that the DPA has a higher affinity to bind to the metal ion than the dye binding to the metal ion for sensitive detection. From this study, terbium and europium were found to be two of the most efficient metal ions in terms of their quenching effect on calcein and ease of dequenching with DPA. Although many others were also shown to have potential. Similarly calcein was found to be one of the most efficient dyes although many others were also shown to have potential.

### Example 2

Calcein and terbium (III) chloride hexahydrate, europium (III) chloride hexahydrate, cobalt (II) chloride hexahydrate, lead (II) chloride, calcium chloride and other general chemicals and solvents were purchased from Sigma-Aldrich (Dorset, England, UK).

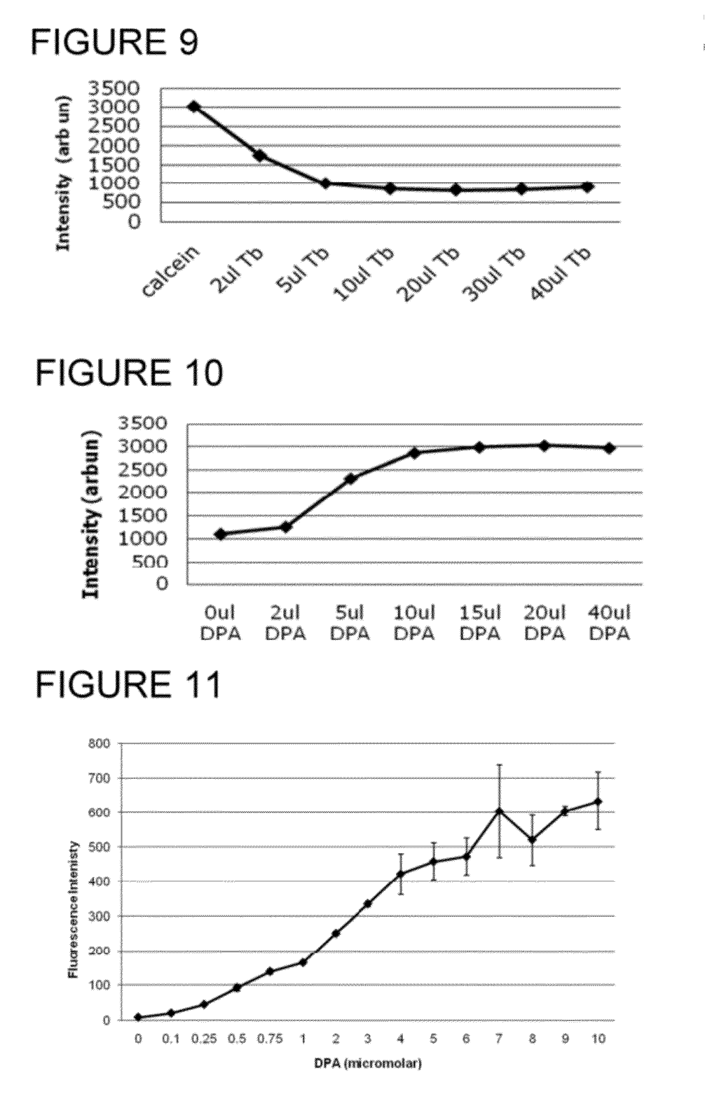

Calcein was selected as the luminescent dye 3, and terbium as the metal ion 4. The fluorescence emission signal 7 was recorded using an excitation wavelength (λex) 82 of calcein set at 485 nm and the emission wavelength (λem) 83 set at 520 nm. FIG. 9 shows the quenching of the fluorescent signal when a fixed concentration of calcein (1.2 μM) is titrated with various aliquots (μL) of terbium chloride (2 mM) solution. As more and more terbium chloride is added, the fluorescence signal drops. From this quenching curve a ratio of greater than four terbium ions per calcein molecule was found to quench the fluorescent signal efficiently. Therefore from such information, quenched solutions of calcein and terbium mixture could be prepared for testing. To reduce the background to a minimum, an excess of terbium chloride could be used in some applications. For instance, from the information in FIG. 9, a quenched solution comprising 1.2 μM Calcein and 19 μM of terbium chloride was prepared by suspending in 10 mM TES 150 mM Sodium Citrate buffer. Then dissolution was achieved by the addition of 5M NaOH until the solution was clear, the pH was returned to 7.4 via the addition of 1M HCl. This quenched solution of calcein and terbium was then used to test the ability of DPA 1 to displace the terbium from the calcein. To do this the quenched solution was titrated with various aliquots (μL) of 2 mM DPA solution. DPA (2,6-Pyridinedicarboxylic acid) was purchased from sigma-Aldrich. FIG. 10 shows the recovery of the calcein fluorescence in response to addition of DPA in μL volumes. Wavelengths used λex=485 nm and λem=520 nm. FIG. 10 demonstrates that as dequenching occurs the fluorescence is recovered (i.e., relieved or re-established) as more and more DPA is added. The fluorescence intensity is particularly high above approximately 10 μL of DPA which is approximately equivalent to the stochiometric amount of terbium used.

### Example 3

A dilute solution comprising calcein and terbium chloride in 1:8 molar ratio, respectively, was prepared in 10 mM TES buffer pH 7.4. Various amounts of DPA were titrated into this solution and changes in fluorescence intensity of calcein measured using an excitation wavelength of 485 nm and emission at 520 nm. FIG. 11 shows the dose response curve for DPA detection.

Further optimization of this assay and reagents including molar ratio and nature of dye and metal used may produce dose response curves with even higher sensitivity.

It is also possible to use the presently disclosed and claimed inventive concept(s) to detect chelators other than DPA. Different chelators may respond differently depending on their binding affinities to the metal. To illustrate how a suitable chelator may be identified the fluorescence emission spectrum of the calcein-terbium stain was recorded in the presence and absence of DPA, EDTA, Lactic acid, 2,6-Diaminopimelic acid (DAP), N-Acetylmuramic acid and acetic acid.

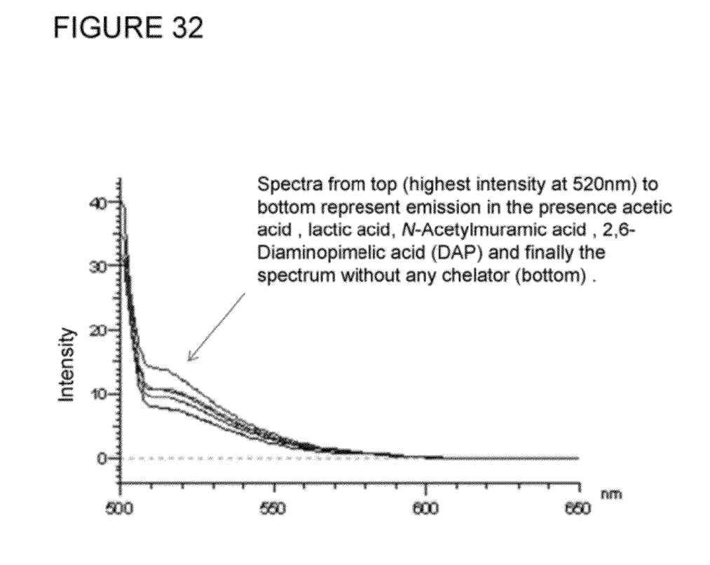

FIG. 31 shows the emission spectrum of 0.12 μM solution of calcein-terbium stain (no chelator spectrum) using an excitation wavelength of 485 nm. When 100 μM of chelator is added the emission spectrum shows a large increase in fluorescence emission for ethylenediaminetetraacetic acid (EDTA) indicating that this could be used as a chelator other than DPA. In contrast, FIG. 32 shows the fluorescence emission increase for other chelators, namely Lactic acid, 2,6-Diaminopimelic acid (DAP), N-Acetylmuramic acid and acetic acid. The increase was only marginal (note the expanded Y-axis compared to FIG. 31), indicating that the detection of these chelators with this particular dye and metal in this particular experiment shows less potential. However it is worth pointing out that a change in assay conditions such as changing the pH may show different potential.

The results of FIGS. 31 and 32 illustrates that the presence of common markers such as acetic acid and lactic acid is not likely to interfere with the detection of DPA which shows relatively large increase in signal intensity at 520 nm compared to lactic acid showing very little increase as shown in FIG. 31 with expanded Y axis.

### Example 4

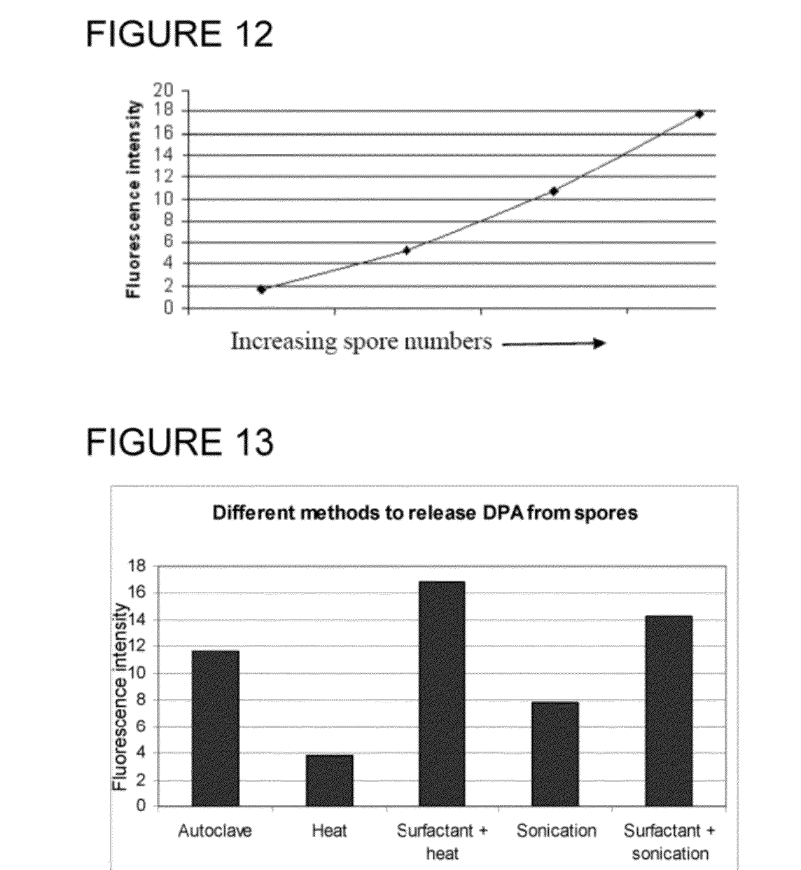

Bacillus cereus spores, ATCC 11778, were purchased (Raven-Laboratories, Inc., Omaha, Nebr., US) in suspended form. Spore suspensions containing different number of spores per mL of 10 mM Tris-pH 8 buffer containing calcein-terbium stain having 0.6 μM calcein were taken and heated at 80 degrees Centigrade for 5 minutes to release some of the DPA from the spores. The suspension was cooled for 5 minutes and then fluorescence was recorded. Wavelengths were set to detect calcein excitation and emission at λex=485 nm and λem=520 nm. FIG. 12 shows recovery of calcein fluorescence in response to increasing number of Bacillus cereus spores. Increasing the spores by 6 orders of magnitude caused fluorescence intensity to double in bulk assay. Signal increase could also be related to DPA content measured by the conventional DPA terbium assay.

The sensitivity of the assay could be further improved by orders of magnitude by optimizing the ratio of calcein to terbium ions, and changing the conditions of the method to release DPA from spores. For example, FIG. 13 shows detection of DPA as indicated by an increase in fluorescent intensity (Y axis) of terbium using the conventional prior-art Terbium chloride assay. In this experiment Bacillus cereus spore suspension received five different treatments, described on the x-axis, to release DPA. For the assay spore suspension equivalent to 3.5×10⁶ spores per mL of solution was used. The treatments were applied for 5 minutes and then solutions allowed to recover for a further 5 minutes before adding to a cuvette having 2 ml of 10 mM Tris-HCL buffer pH 8 containing 30 μM terbium chloride. For sonication a bath (Ultrawave Ltd, Cardiff, UK) was used, for heat treatment spore samples were incubated at 80 degrees centigrade for 5 minutes. Dodecylamine was used as a surfactant at 1 mM concentration. When dodecylamine was used in combination with heat or sonication, it was added before applying heat or sonication. In all cases, the final reading was after 10 minutes from adding spores to the terbium chloride solution. The terbium fluorescence was recorded at 550 nm with an excitation wavelength of 278 nm. This conventional assay shows that DPA release can be mediated by various treatments. Such methods could be used to release DPA which may then be detected by a dye-metal stain such as described with reference to FIGS. 1 to 3.

### Example 5

Detection of DPA within spores:

A solution of calcein and terbium was prepared comprising 1.2 mM calcein and 10 mM terbium in 10 mM Tes pH 7.4 buffer. This is the stock stain solution which from here on is referred to as the 1.2 mM Calcein-terbium stain or simply stain. For microscopy experiments the final concentration used was 1.2 μM of this stain. Such solutions were then used as a calcein-metal stain to visualize spores microscopically by the method below.

Bacillus cereus spores, ATCC 11778, were purchased (Raven-Laboratories Inc., Omaha, Nebr., US) in suspended form and diluted to 1×10⁷ spores per milliliter. An aliquot of the spore suspension was placed on a microscope slide using an inoculation loop. This was fixed to the slide by air drying. The slide was flooded with a solution of the calcein-terbium stain (1.2 μM) prepared above. Another slide was prepared in the same way but this time it was gently heated over a Bunsen flame. Both slides were washed with deionised water to remove any excess unbound material. The slides were dried and examined under a Leica SP5 upright microscope. Images were recorded under brightfield mode and fluorescence mode. For fluorescence images Fluorescein Isothiocyanate (FITC) filters were used as they have similar excitation and emission wavelengths to calcein.

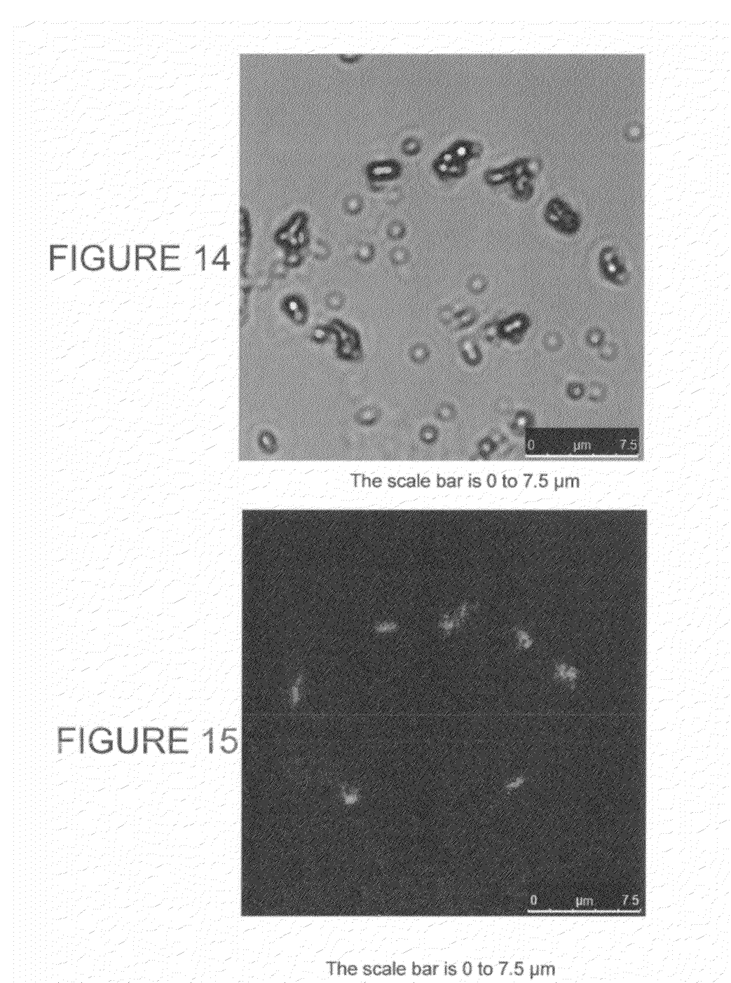

Results are shown as micrograph images in FIGS. 14 and 15. FIG. 14 shows the brightfield image in which the spores have been stained and then fixed to slide using heat; the spores are clearly visible as white dots. FIG. 15 shows a fluorescence image obtained from the same sample obtained using FITC filter settings on the same Leica microscope. The green spots (which in the fluorescence microscope image appeared as intense green spots) have high green fluorescence which coincide with the spore structures shown in FIG. 14 when the figures are overlaid. The fluorescent spots are thus indicative of the spores. By “high” it is meant that the fluorescence is readily discernible from the background by a human eye when using a microscope.

It is noteworthy in FIGS. 14 and 15 that not all the spores stain to the same intensity. Without intending to limit the scope of the presently disclosed and claimed inventive concept(s), it is believed that spores having higher DPA content stain more strongly than those with less DPA content. Comparison of brightfield image (in FIG. 14) with the fluorescence image shows that from the intensity of the fluorescence (in FIG. 15) it is possible to distinguish between spores that are viable in that they have a normal or high DPA content and those that are non-viable which have low or zero DPA content. The intensity level of the fluorescence signal that corresponds to a viable spore can be determined by experiment. The intensity level that corresponds to a viable spore may be different for different types of spore.

Spores were also observed using the calcein-terbium stain without using heat on the microscope slide. In these cases, the Bunsen flame was not used to fix the spores to the slide but instead the sample was allowed to dry as described above. The fluorescence photomicrograph image of this sample (using FITC filters) is shown in FIG. 17. The green spots (which in the fluorescence microscope image appeared as intense green spots) have high green fluorescence and indicate the detection of DPA in spores. It is thus possible to sensitively observe spores without having to apply stimuli to access the DPA in the spore. That is, it is possible to penetrate the spore with the calcein-metal stain.

Spores from other bacteria could also be detected similarly. As an example for instance FIG. 21 shows the fluorescence image for Clostridium difficile spores when detected microscopically using calcein-terbium stain (1.2 μM) with identical method to that described for Bacillus cereus spores (FIG. 17). Again the green spots (which in the fluorescence microscope image appeared as intense green spots) have high green fluorescence indicating the presence of spores. Spores in the presence of vegetative or mother cells can be detected. To show this, endospores were prepared from sporulating culture of Bacillus cereus. After growth of these microorganisms on nutrient agar at 37 degrees Celsius for 24 hours, the cell mass was scrapped and suspended in water and spun at 5000 rpm for 5 minutes. The supernatant was then removed and the pellets washed two more times with water. The final pellet was resuspended in water and was used without any further attempts to remove vegetative cells. An aliquot of the spore suspension (1 μL) was placed onto a microscope slide. The slide was air dried and then flooded with Calcein-Europium stain for 1 hour. The excess stain was then washed off with deionised water, and the slide air dried. The fluorescence image was recorded on the SP2 inverted confocal microscope. Such beads as internal calibrants or internal size references can be advantageously used in accordance with the other aspects of this presently disclosed and claimed inventive concept(s).

Images were recorded under fluorescence mode using FITC filters and settings. FIG. 27 shows a fluorescence image obtained from this sample. The brighter green spot indicated by the arrow (which in the fluorescence microscope image appeared as intense green spot) has high green fluorescence and is recognised as an endospore inside a cell or sporangia which appear as rod like structure that does not stain as well as the endospore since it has no DPA.

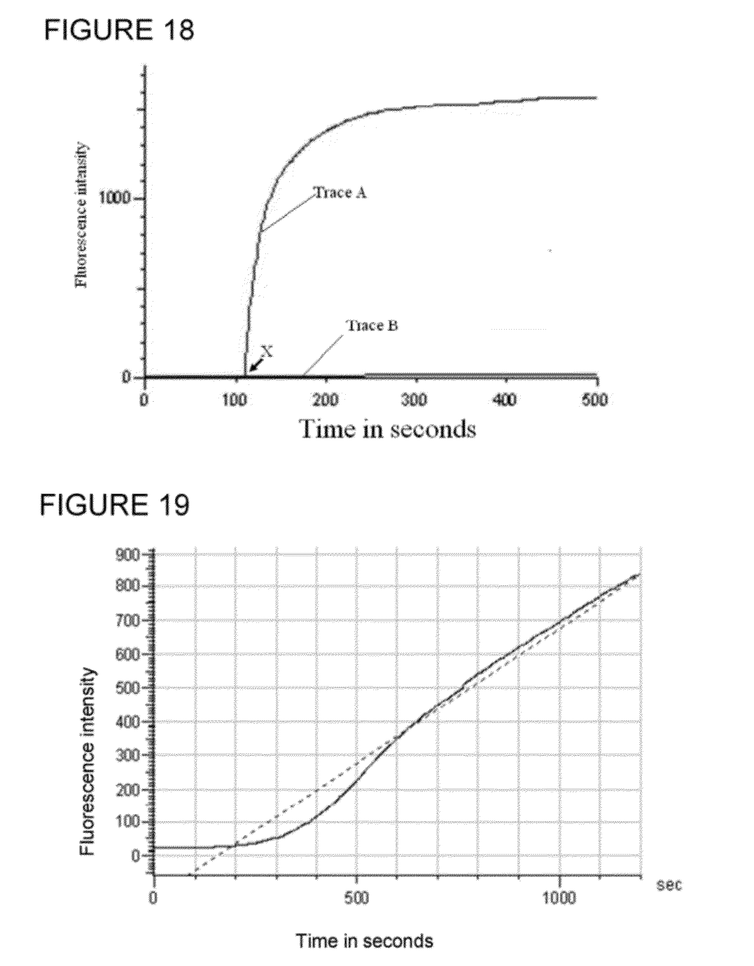

An experiment was conducted using spectrofluorometry to show that a metal-dye stain is uptaken by the spores without any treatment in solution. In this experiment Bacillus cereus spores (1×10⁷) were added to a 1.2 μM solution of calcein-terbium stain and changes in fluorescence emission intensity followed as a function of time. The excitation and emission wavelength were 485 nm and 520 nm. FIG. 19 shows a plot of fluorescence intensity against time for this experiment. An increase in signal intensity is observed with time. The data in FIG. 19 shows that calcein-metal stain was able to penetrate the spores and interact with DPA allowing detection. The spore DPA is able to displace the metal from calcein-terbium to dequench the fluorescence. As time proceeds more and more dye-metal penetrates the spores giving higher fluorescence intensity. Thus the dye-metal stain can be used to detect DPA from intact spores. A similar experiment using just calcein showed no observable change in fluorescence thus indicating that the signal is specific to the dye-metal stain detecting DPA. To substantiate this further another experiment was conducted to demonstrate that the dye-metal stain is responsive to DPA but not to calcium. For this experiment a solution of calcein-terbium stain (1.2 μM) in 10 mM TES, 100 mM NaCl pH 7.4 was taken. Fluorometer was set to excitation wavelength of 485 and emission at 520 nm. Fluorescence intensity was recorded as a function of time. After 2 minutes (indicated by an arrow on FIG. 18) excess DPA (100 μM) was added and fluorescence measurements continued. The same experiment was repeated but this time adding calcium chloride (100 μM) instead of DPA was added. Results are shown in FIG. 18. It is clear that calcein-terbium stain has very low background that remains steady prior to adding DPA (FIG. 18, Trace A). Upon adding DPA the fluorescence intensity increases rapidly and is established. Therefore the dye-metal has responded by re-establishing or dequenching the fluorescence and thus DPA is detected. When considering Trace B (FIG. 18) no increase in fluorescence intensity is seen above the background indicating that the dye-metal stain is relatively insensitive to calcium. Note that background signal prior to (before time point X FIG. 18) any addition of DPA or calcium chloride overlap and also being so low are in fact overlaying the x-axis.

### Example 6

In further demonstration of the presently disclosed and claimed inventive concept(s) a suspension of Bacillus cereus spores was treated with the stain used in example 5. An aliquot of this was analysed by flow cytometry showing spore specific staining with green fluorescence.

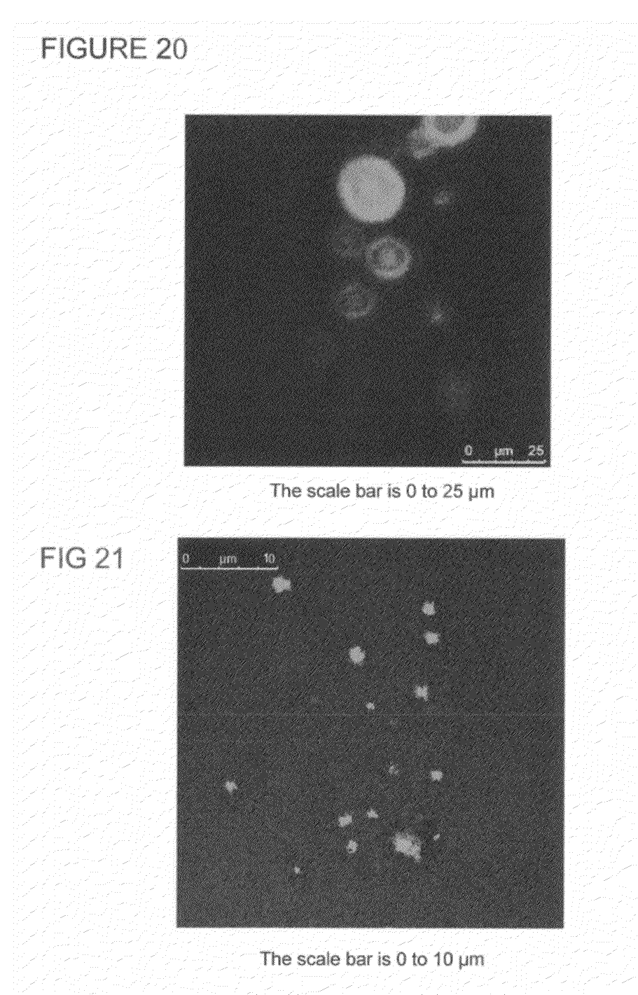

In yet another demonstration, Bacillus cereus spores were captured on magnetic bead and stained. The beads were prepared by incubating an antibody (ThermoFisher Scientific, Waltham, Mass., US) to Bacillus cereus with protein G dynal beads (obtained from Invitrogen, Grand Island, N.Y., US) in Phosphate Buffered Saline (PBS) and washing the excess antibody off by capturing the beads with magnet and washing off the solution. The beads were finally suspended in 10 mM Tris pH 8 buffer at a concentration of 1×10⁸ per ml. In this experiment 5 μl of these magnetic beads (2.8 μm) were added to spores (1×10⁶) suspended in 100 μl of buffer and incubated for 40 minutes followed by three wash steps using magnetic capture. The sample was then re suspended after the final wash in 20 μl of buffer and stained using calcein-terbium stain (1.2 μM) for 10 minutes and imaged by fluorescence microscopy as before in Example 5. FIG. 20 shows the fluorescence image obtained using FITC filter settings on the Leica SP5 upright microscope. Rings of green (which in the fluorescence microscope image appeared as intense green rings) having high green fluorescence appeared around the magnetic beads. The results thus show that that fluorescence accumulates preferentially around the magnetic beads. It is thus possible to detect specific spores using antibodies in combination with the dye-metal in the presently disclosed and claimed inventive concept(s). Other methods of immunocapture may also be used. Similar results may be obtained for other spores including C. difficile and using different immunocapture beads such as including latex beads. Such immunocapture beads can be advantageously used with the other aspects of this presently disclosed and claimed inventive concept(s). Filtration and centrifugation may also be used as techniques to capture spores. For instance centrifuging at 5000 g for 5 minutes and collecting the pellet concentrates the spores and filtering a suspension of spores via a 0.22 micron will concentrate the spores on the filter as they are too large to pass through. For instance, FIG. 29 shows photomicrograph of Bacillus spores from centrifuge pelleted sample when stained by calcein-europium stain ((as prepared in Example 8) and observed as stated in example 8 using an inverted epifluorescence trinocular microscope (Fisherbrand, Cat No FB69198) at 400 times magnification. The intense green spots (which in epifluorescence photomicrograph appeared as intense green spots) have high green fluorescence and indicate the detection of DPA in spores. It is thus possible to concentrate spores and recover them for microscopic imaging using europium based calcein stain and also without having to apply stimuli to access the DPA in the spore.

Once the stain has been uptaken by the spore, it may be possible to add further metal ion to the solution in order to further quench any non-specific signal outside the spore. For instance cobalt chloride solution may be used for this purpose. It is also possible to use such solution as a wash solution for washing spores to remove non-specific background. Example 2 above shows that cobalt ions among many others can quench the dye to reduce fluorescence emission.

### Example 7

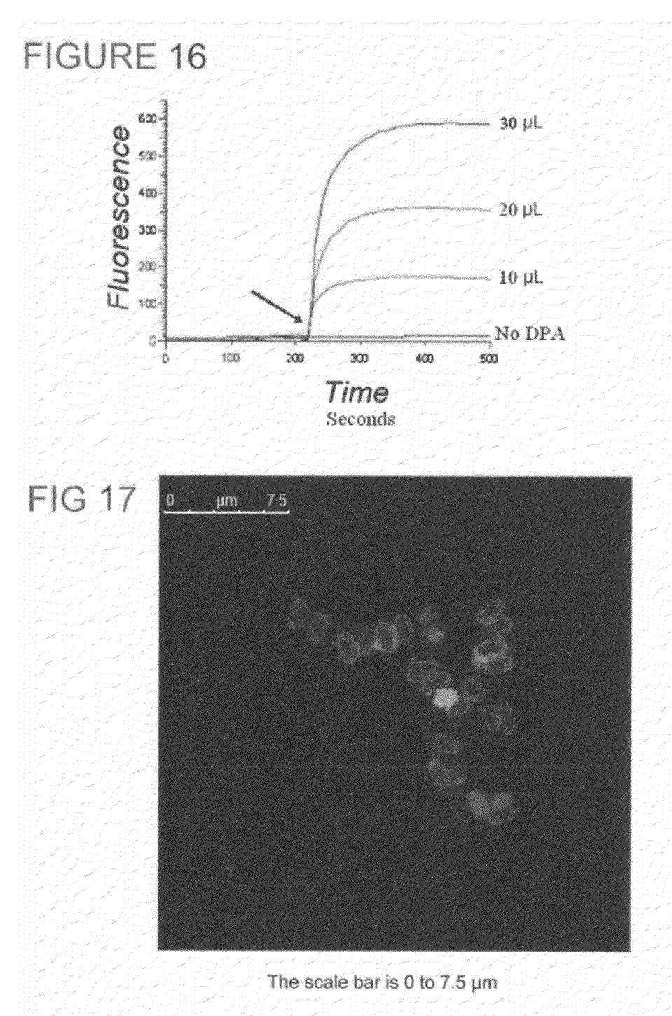

Unilamellar liposomes were used to encapsulate the calcein-terbium stain described above with reference to example 5. These were prepared by hydrating a lipid film, comprising 2:1 molar ratio of phosphatidylcholine (Lipid Products, Nutfield, UK) and cholesterol (Sigma) with 12 mM calcein dye containing 100 mM terbium chloride (Sigma) in 10 mM Tris pH 8 buffer. The hydration mixture was subjected to ten extrusion cycles through (Avestin Liposofast 100) 200-nm pore polycarbonate filters (Nucleopore) at room temperature. The non-encapsulated calcein-terbium stain was removed by gel filtration, through a Sepharose-6CLB (Pharmacia), 1.5 cm×25 cm column using iso-osmotic eluting buffer. Liposomes prepared this way remained stable and were diluted to 3 mg/ml lipid concentration. Four assays (labelled “no DPA”, 10 μL, 20 μL and 30 μl of a 2 mM stock) were conducted as follows. A 3 μL aliquot of liposomes was added to 10 mM Tris pH8 buffer and fluorescence emission recorded as a function of time using an excitation and emission wavelengths of 485 nm and 520 nm respectively. After a short period of time indicated by the arrow (FIG. 16) 104 of 10% sodium deoxycholate was added to rupture the liposomes. The assay was repeated but with buffer solution containing different volumes (10 μL, 20 μL and 30 μl) of 2 mM DPA solution added. While monitoring the fluorescence from the calcein, the liposomes were ruptured using detergent in order to release the stain from the liposomes. The results are shown in FIG. 16 which shows the relative fluorescence versus time in seconds for the four different assays. The arrow indicates the time in which the detergent was added to the solution. Upon rupture the stain is released and able to bind with DPA to show fluorescence increase. The results demonstrate that the increase in fluorescence is proportional to the level of DPA in the solution. Other experiments have revealed that cytolytic peptides such as melittin can also be used to release the stain from the liposomes.

In further work, such liposomes were sensitized with antibodies to Bacillus cereus spores purchased commercially (Thermo Scientific). The liposomes could be made to release DPA in the spore suspension staining B. cereus spores. In this manner, specific spores may be detected by staining the spores that have bound the liposomes in preference to those that have not bound the liposomes.

The results demonstrate that it is possible to target the calcein-terbium stain to spores for specific detection. For instance this can be done using receptors, ligands or antibodies to specifically target the spore surface. It is also possible for the stain to be encapsulated in particles or other matrices and deliver it to a specific spore. FIG. 16 shows that the stain can be encapsulated in 200 nm liposomes and released using a cytolytic peptide or detergent. Upon release the calcein-terbium stain is able to bind to DPA and show signal enhancement. Such systems may be used in staining specific spores. The results also demonstrate that the stain and DPA can be contained in different compartments without reaction as little or no change is seen before (<200 seconds FIG. 16) adding detergent. In this case the calcein-terbium stain is in the lumen compartment of the liposomes while DPA is free in solution. Rupture of liposomes or penetration of compartment with peptide allows the DPA to reach the stain giving signal. The situation could be compared to provide a model of spore detection, in converse sense, where the stain is free in solution and DPA is in the compartment (core) and by penetration the two meet to give signal.

Another example of the way to target the spores is to capture spores on immunomagnetic beads tethered with liposomes encapsulating the dye-metal. Alternatively one could target the stain to the spore surface or its coat protein. A number of methods are commonly available to conjugate dyes to targeting molecules including antibodies. The number of spores may be captured on such beads and beads treated with the stain to reveal the signal around the bead. Targeting approaches involving micro or nano sized particles are available in addition to direct molecular targeting, and these are included within the scope of the presently disclosed and claimed inventive concept(s).

### Example 8

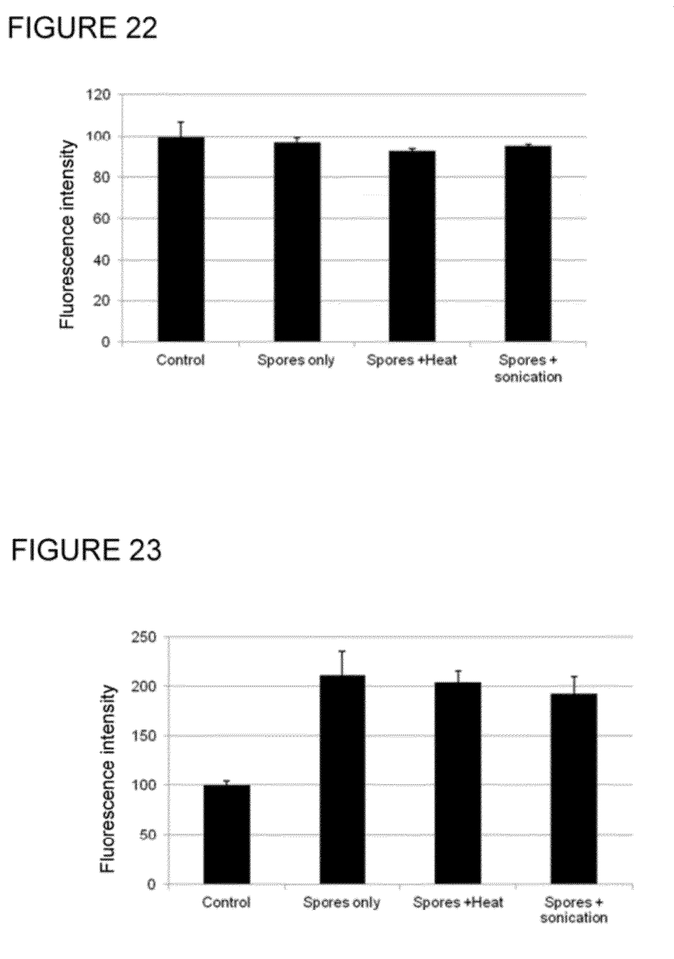

To demonstrate that dye-metal stain is able to detect whereas the dye alone without the metal is unable to detect DPA from the spores, the following experiment was carried out. For the assay, Bacillus cereus spore suspension equivalent to 3.5×10⁶ spores was used. Various treatments were applied for 5 minute and then solutions allowed to recover for further 5 minutes before adding to a cuvette having 2 ml of 10 mM Tris-HCL buffer pH 8 containing either 1.2 μM calcein-terbium (dye-metal stain FIG. 23) or, in the case of dye alone, 1.2 μM calcein (FIG. 22). For sonication bath (Ultrawave Ltd) was used, for heat treatment spore samples were incubated at 80 degrees centigrade for 5 minutes. In all cases final readings were taken after at 60 minutes from adding spore suspension to the calcein solution. The calcein fluorescence was recorded at 520 nm with an excitation wavelength of 485 nm. For the control experiment no spores were added. It is clear from FIG. 22 that no significant change in fluorescence intensity relative to the signal in absence of spores was seen (FIG. 22) for calcein alone indicating that calcein alone is not able to detect DPA from spores in solution assay. Furthermore treating spores with ultrasound or heat did not show any detection either when calcein alone is used. In contrast there is significant increase in fluorescence intensity (FIG. 23) when calcein-terbium is used in the presence of spores compared to when spores are absent. Furthermore the strong comparable signal is achieved without applying any heat or ultrasound treatment to the spores when using calcein-terbium stain.

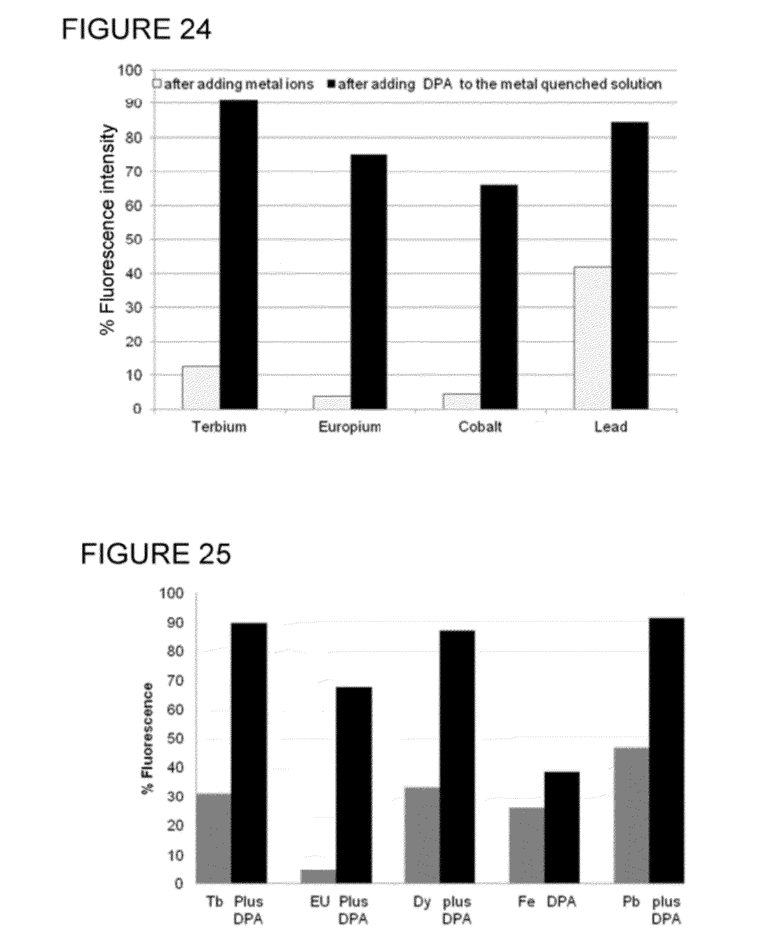

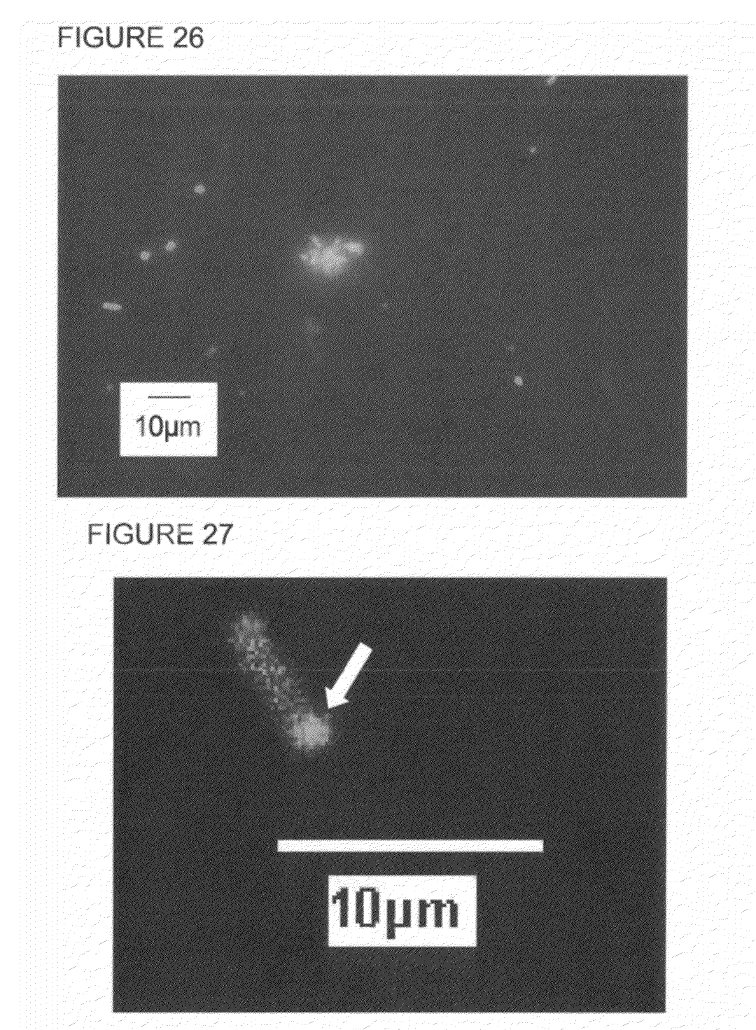

As mentioned earlier other metal ions than terbium can be used to detect DPA according to the presently disclosed and claimed inventive concept(s). For instance, FIG. 24 compares the fluorescence intensity of calcein with few other selected metals in the presence and absence of DPA. In these experiments calcein solution (1.2 μM) was taken and its fluorescence intensity recorded. Then metal ions (10 μM) were added and solution mixed and fluorescence intensity recorded again. Finally excess DPA (500 μM) was added and fluorescence measured. On the y-axis is shown % fluorescence intensity relative to calcein alone. This was calculated by dividing the observed fluorescence intensity signal by the fluorescence intensity signal of calcein alone (without the metal) and then multiplying by 100. Clearly the calcein is quenched by metal ions europium, cobalt, and lead and dequenching occurs when DPA was added to this metal quenched solutions thus showing that these metal ions could also be used to detect DPA just like the terbium. For instance a stain was prepared using europium metal rather than terbium. To make 1 ml of calcein-europium stain 0.25 ml of 1 mM Calcein dye is mixed with 0.5 ml of 1 mM europium (III) chloride and a further 0.25 ml volume of buffer is added. All solutions are in 10 mM Tris-HCL buffer pH 7.5. To demonstrate that this stain is capable of detecting spores Bacillus cereus spores were stained. A sample (5 μl) of the spores was applied to a microscope slide, and allowed to air dry for 1 hour. Then 5 μl of the calcein-europium stain was added and dried for 1 hour, the excess stain was washed off with deionised water and the slides dried. Slide was then viewed on an inverted epifluorescence trinocular microscope (Fisherbrand Cat No FB69198) at 400 times magnification and using blue fluorescence excitation dichroic filter Cube (excitation spectrum 420-485 nm) and green emission filter 515 nm. FIG. 26 shows an epifluorescence photomicrograph of the stained spores. The intense green spots (which in epifluorescence photomicrograph appeared as intense green spots) have high green fluorescence and indicate the detection of DPA in spores. It is thus possible to sensitively observe spores using europium based calcein stain and also without having to apply stimuli to access the DPA in the spore. That is, it is also possible to penetrate the spore with the calcein-europium stain. Other dyes could be similarly tested for their competitive binding to DPA and used in the presently disclosed and claimed inventive concept(s). For instance FIG. 25 shows results for 1.5 μM Phen Green (invitrogen) solution in 10 mM Tris-HCL pH 7.5 buffer. Excitation and emission wavelengths were 507 nm and 532 nm respectively. Grey bars are after adding the metal ion (10 μM) to the Phen Green solution and the black bars are when DPA (0.5 mM) is added to this same solution containing the dye and metal ions. On the y-axis is shown % fluorescence intensity relative to Phen Green alone. This was calculated by dividing the fluorescence intensity observed by the fluorescence intensity of calcein alone (without the metal) and then multiplying by 100. Clearly fluorescence can be recovered in the presence of DPA indicating that such dyes could also be used.

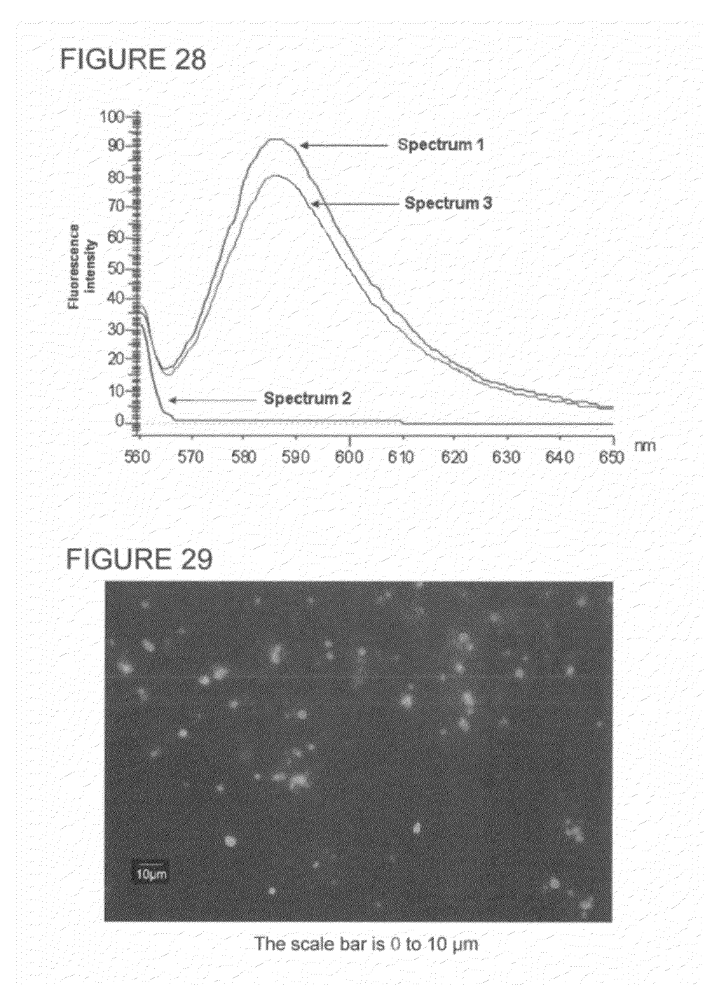

In similar experiments resorufin is another dye which can be quenched with number of metal ions metal ions and upon adding DPA to this dye metal solution fluorescence increase observed. For instance FIG. 28 shows the emission spectrum of 1.6 μM solution of resorufin (spectrum1). When 10 μM europium is added to this same solution the resulting spectrum 2 was obtained indicating quenching of fluorescence as seen by reduction in signal intensity between 580 to 600 nm. When 50 μM DPA was added to the quenched solution the fluorescence emission recovered as shown by spectrum 3. In these cases the spectra were recorded in 10 mM Tris-HCL pH 7.4 buffer and fluorescence measurements were made on a Hitachi F-2500 fluorescence spectrophotometer with a 1 cm path length cuvette and an excitation wavelength of 550 nm. The data illustrates that resorufin-europium stain is also capable of detecting DPA.

The presently disclosed and claimed inventive concept(s) could be made to work in the reverse sense whereby a metal ion may enhance the fluorescence of the dye and DPA then acts to reduce it by binding to the metal ion. This way the signal is opposite to the signal in the previous examples of the presently disclosed and claimed inventive concept(s) in that the background will stain more than the spores. This method of negative staining can also be used to detect the spores. These negative methods, reagents and apparatus are included with the scope of the presently disclosed and claimed inventive concept(s).

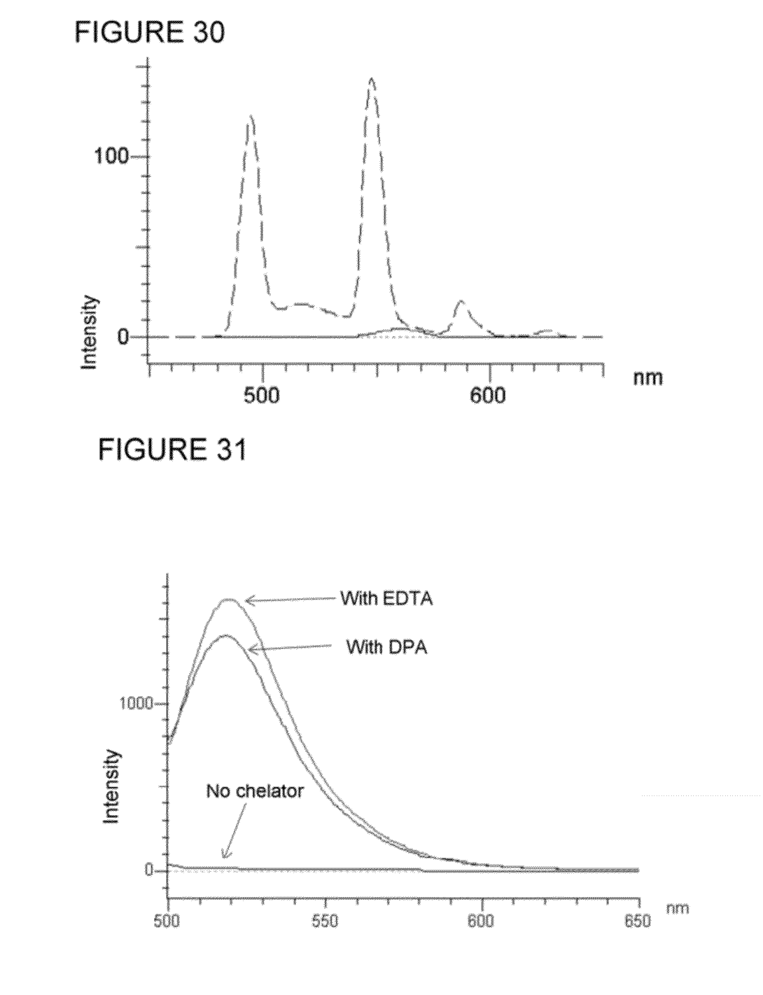

It is also possible to use the previously described methods of the presently disclosed and claimed inventive concept(s) by exciting the fluorescence of the dye by using a wavelength of light that optimally excites a lanthanide-ion chelate comprising the lanthanide ion and the DPA 1. This way signals from the lanthanide-ion chelate can be measured, signals from the dye can be measured, or fluorescence signals from both the lanthanide-ion chelate and the dye can be measured. To illustrate this, a 1.2 μM calcein-terbium stain, 1 μl aliquot was added to a cuvette contain in 1 ml of buffer (10 mM TRIS pH 7.4) fluorescence emission scanned on an Hitachi F-2500 fluorescence spectrophotometer, with setting of λEx 277 nm and λEm 450-650 nm Emission spectrum, represented by solid line, in FIG. 30 is shown for this solution. Then an aliquot of 10 μl DPA (10 mM) was added and the scan performed again after 1 minute. The emission spectrum of this mixture is shown in FIG. 30 by dotted line. When DPA is added the increase in emission intensity at 490 nm indicates the formation of the Terbium-DPA complex while the increase at 520 nm shows the displacement of metal ion from calcein. It should be noted that the excitation wavelength used is not the most appropriate for efficient calcein excitation. However key point is that both the calcein and the terbium fluorescence can be measured individually, separately, or simultaneously in response to addition of the DPA. The use of dual fluorescence may thus provide added selectivity for detection of DPA as the calcein signal originates from displacement of the terbium with a concurrent increase in fluorescence of the terbium DPA complex. This could be used to confirm that the response seen from calcein signal is due to DPA detection aiding selectivity of the detection of DPA compared to detection of other chelates (if present).

### Example 9

Example 9 has been chosen to illustrate how a luminescent dye can self quench above a certain concentration, and how the self quenching can be relieved by reducing the concentration of the dye. This property is used in Example 10 to monitor a sterilization process.

Calcein dye was encapsulated inside unilamellar liposomes. The liposomes were prepared as in Example 7, except that calcein dye was used instead of the calcein-terbium stain of Example 7. These unilamellar liposomes were 200 nm in diameter and had calcein at 120 mM concentration encapsulated inside, which concentration is well above the known concentration levels at which calcein self quenches (approximately 1 μM calcein concentration).

A 3 μL aliquot of the liposomes were added to 2 ml of 10 mM Tris pH8 buffer and the fluorescence emission recorded as a function of time using excitation and emission wavelengths of 485 nm and 520 nm respectively. After a short period of time 10 μL of 10% a detergent (TRITON™ X-100) was added in order to rupture the liposomes and release the calcein from the liposomes into the solution.

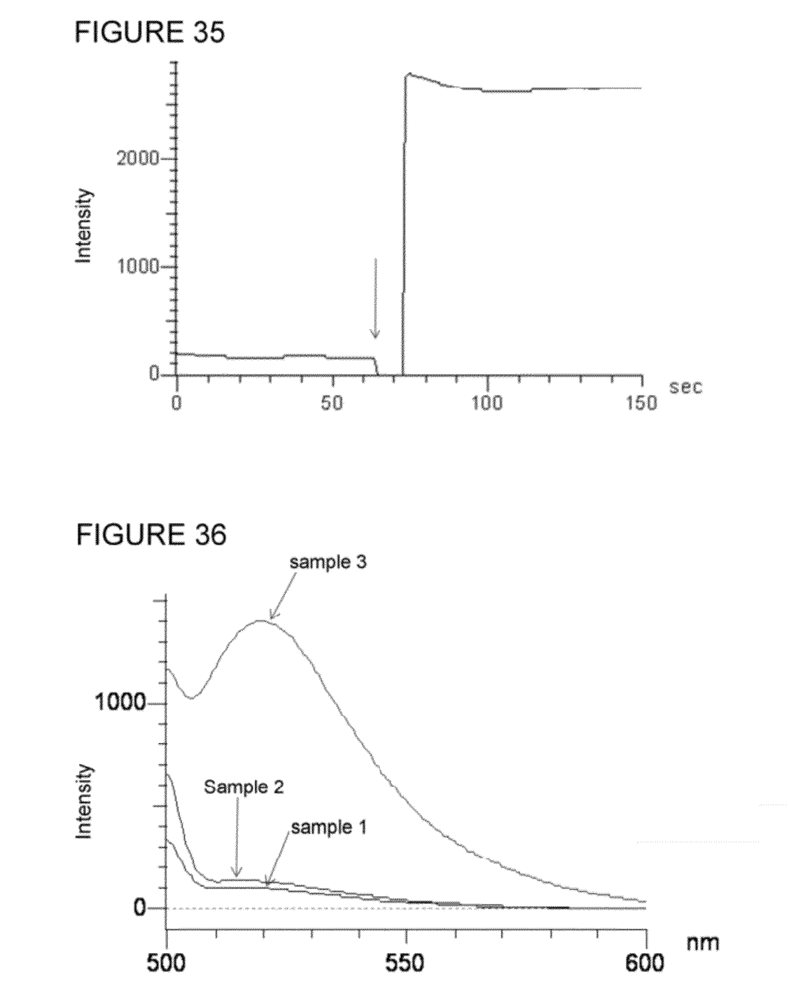

The relative fluorescence versus time in seconds was recorded during the experiment, and is shown in FIG. 35. The arrow indicates the time when the detergent was added to the solution. Prior to adding the detergent, the relative intensity of the fluorescence was low, indicating that, as expected, the calcein had self quenched inside the liposomes. Upon rupture, calcein was released from the liposome lumen into the surrounding buffer and the self quenching was relieved as evidenced by the increase in the fluorescence intensity. The results demonstrate that the increase in the fluorescence is due to dilution of the dye from an initial concentration of 120 mM inside the liposomes, to less than a micromolar concentration in the solution. The concentration of calcein in solution is less that the self-quenching concentration of calcein which is why the fluorescence intensity increased.

Although this example has been carried out with calcein, many other luminescent dyes also self quench.

### Example 10

It is known that the concentration of DPA inside endospores can in the millimolar (mM) range although it may vary depending on the bacteria. In any case it is usually well above 1 micromolar (1 μM), which is the self-quenching concentration threshold of the calcein dye. Note that for a self quenching dye a plot of the luminescence intensity against concentration of the dye typically exhibits a bell shaped curve which shows that the luminescence intensity rises till a certain threshold concentration is reached after which the luminescence intensity drops. This is relevant because luminescent dyes can be introduced into the endospores as a dye-metal complex. The dye is released from the metal ions by the DPA molecules. And thus the concentration of the dye within the endospore can be increased to the concentration level of the DPA. Since this concentration is much greater than the self quenching concentration of the dye, it is possible to manufacture spores containing self-quenched dyes, and use these to monitor the sterilization and/or disinfection process.

It is also known that DPA can be released from endospores by using physical or chemical treatment. Such processes can also be used to release the calcein dye which has been preaccumulated into the endospore. As the dye is released into a surrounding solution, the fluorescence of a pre-quenched sample is expected to increase.

An aliquot of Bacillus cereus spores (60 μl) was placed into a glass vial containing 240 μl buffer (10 mM Tris-HCL pH 8) and 120 μl Calcein Europium stain (100 μM). The Calcein Europium stain was such that (i) the Calcein had been quenched by the Europium ions, and (ii) the concentration of the Calcein Europium stain was far in excess of 1 μM, the self-quenching concentration threshold of Calcein (without the metal ions). The vial was incubated at room temperature for 1 hour in order to allow sufficient reagent to penetrate the spores, for the DPA to release the Calcein dye from the Europium ions, and for the Calcein dye to become self quenched. The sample was split into two vials each containing 210 μl volume. One vial (sample 1) was kept on the bench, while the other (sample 3) was placed into an autoclave together with a vial of 120 μl of the Calcein Europium stain (sample 2) diluted in 150 μl of the buffer. The sample 2 and 3 vials were autoclaved simultaneously at 120 degrees centigrade for 30 minutes, then removed, allowed to cool, and the volumes in all three vials were reconstituted to 300 μl with deionised water. An aliquot of 50 μl of the each was then placed into a cuvette containing 1 ml buffer (10 mM TRIS pH 7.4) and a fluorescence emission scan preformed from 500-600 nm using an excitation wavelength λEx of 485 nm.

FIG. 36 shows the emission spectrum obtained for each sample. The pre-quenched spores, sample 1, showed a low fluorescence emission intensity at 520 nm. In this case the fluorescence is quenched due to self quenching of the dye inside the spores and metal quenching of any dye outside the spores. Upon autoclaving the pre-quenched spores (sample 3) the dye that has been displaced from the metal by DPA is released from the spores into the buffer where the dilution of the dye concentration relieves the self quenching resulting in a much higher fluorescence emission as seen for spectrum 3. The control experiment (sample 2) using only the stain (with no spores) only exhibited a low fluorescence emission at 520 nm, indicating that the stain is stable to the autoclaving process. The increase in intensity when autoclaving the pre-quenched spores (sample 3) is the result of the dye-metal complex becoming in contact with the inside of the spore contents, including exposure to DPA.

Thus pre-quenched luminescent spores can be prepared using a concentration of stain that exceeds its self-quenching threshold. The stain is loaded into the spore and calcein displaced form the metal. However, the free calcein, rather than becoming luminescent, becomes quenched (self quenching) due to the high concentration of DPA (greater than the self quenching of calcein) in the spore. Thereafter by applying heat the calcein from inside the spore is released and upon dilution into the surrounding media the fluorescence increases by relief of self quenching. Hence sterilization (such as autoclaving) and disinfection can be monitored by observing the increase in fluorescence from a sample containing pre-quenched spores.

Examples 9 and 10 demonstrate how the presently disclosed and claimed inventive concept(s) can be used to monitor sterilization and/or disinfection processes by measuring a change in the luminescence of a self-quenched dye as it becomes unquenched due to the relief of the self-quenching. The self quenching may be relieved by dilution of the dye from a concentration at which the dye self-quenches to a lower concentration at which the self quenching is relieved and at which the dye becomes luminescent again. With reference to FIG. 33, the dilution may be achieved by exposing the self-quenched endospores 332 that contain the dye to a sterilization and/or disinfection process. The endospore 332 can be prepared with luminescent material 333 at a concentration inside the endospores sufficient to cause self quenching and thus a relatively low luminescent signal 7. Upon sterilization and/or, the barrier 335 is breached, allowing the luminescent dye to escape from the endospore 332 into its surroundings (typically a fluid). The luminescent dye becomes sufficiently diluted to produce a higher luminescence signal from within the endospore. Alternatively or additionally, the concentration in the surrounding fluid also contains a dye at concentrations at which the luminescence of the dye is relieved, and thus provides a luminescent signal.

Although this example has been carried out with calcein and Europium, many other luminescent dyes also self quench. The presently disclosed and claimed inventive concept(s) will thus also work with many other dyes and metal ions as described herein.

The stain containing endospores could also be examined by microscope before and after sterilization. Whether the endospores become more stained after autoclaving may depend on the level of DPA inside the endospores as well as other characteristics affecting stain penetration. Such endospore characteristics include, but are not limited to, the strain type, age, history of storage conditions, and viability status. Thus, a very high DPA may displace the metal from the stain, resulting in more free dye that may self quench the signal more if the dye used has self-quenching properties. Thus, in certain circumstances, it may be possible for a low level of DPA to give a higher intensity signal, as less self quenching of free dye may occur inside the spores. It is therefore possible that, by limiting the amount of stain used, one can make the endospores become either more or less luminescent when subjected to sterilization.

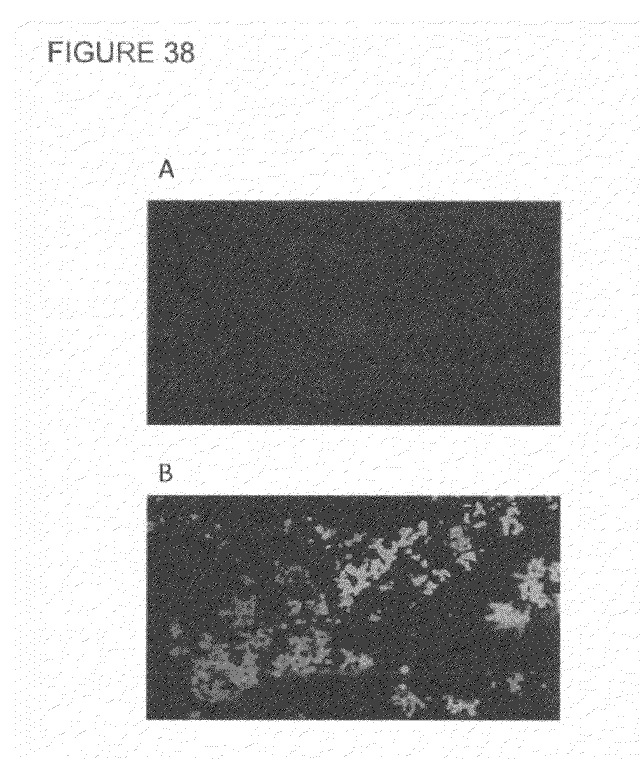

Aliquots of Bacillus atrophaeus endospores (NAMSA, Northwood, Ohio, USA, 1.6×10⁶ per 0.1 ml), commonly used for testing autoclaves, were placed onto two separate slides and then allowed to dry. After drying, the slides were washed with deionized water and dried again. An aliquot of 50 μl of 1:10 dilution of the europium stain (50 μM Eu) was added to the slides and incubated for 45 minutes at room temperature, after which the slides were washed (with 3×1 ml aliquots of deionized water) and air dried. Thus, at this stage, the endospores were expected to have been pre-loaded with stain. One slide was left on the bench, as a control, while the second slide was placed into an autoclave and subjected to one standard sterilization cycle for 15 minutes at 120° C. Upon completion of the autoclave cycle, the slide was cooled to room temperature and imaged by using an inverted fluorescence microscope. FIG. 38 shows a photomicrograph of these endospores using an inverted EPI-fluorescence trinocular microscope (Fisherbrand Cat No FB69198) at 100× magnification. The intense green spots in FIG. 38 have high green fluorescence and indicate the detection of DPA in spores. It is clear that the stain loaded autoclaved endospores have much higher intensity (panel B), and have thus been made luminescent, compared to the non-autoclaved (Panel A) sample. The reasons for higher intensity may be that the endospore compartments have become labeled with the free dye displaced by DPA, and, upon autoclaving, the overall concentration of the dye may drop below the self quenching concentration to generate a bright signal for these endospores.

### Example 11

Examples 9 and 10 demonstrated the presently disclosed and claimed inventive concept(s) using self-quenched luminescent spores. The presently disclosed and claimed inventive concept(s) also works for spores that have not been self-quenched as will be demonstrated in this example.

An aliquot of 100 μl of Bacillus cereus spores were placed into a vial containing 200 μl deionised water and autoclaved at 120 degrees centigrade for 15 minutes. Then a 2.5 μl of the autoclaved sample was placed on a microscope slide which was dried by exposing to air for 1 hour and then washed with water. Thus at this stage, the spores may have some of their DPA leaked into near vicinity. The slide was then treated with calcein-europium stain for 1 hour as before (example 8) to stain the spores before a final wash and drying. It would be expected that the stain will become loaded into the spores to form free dye that depends on the amount of DPA present. Thus, the autoclaved sample will have lower DPA than the non-autoclaved sample, as heat is known to release DPA. The slide was viewed on SP2 Lecica microscope under fluorescence mode at 400× magnification. To have comparable data for the non-autoclaved sample, another slide was prepared using same batch of endospores and in an identical manner except that the autoclaving process was omitted.

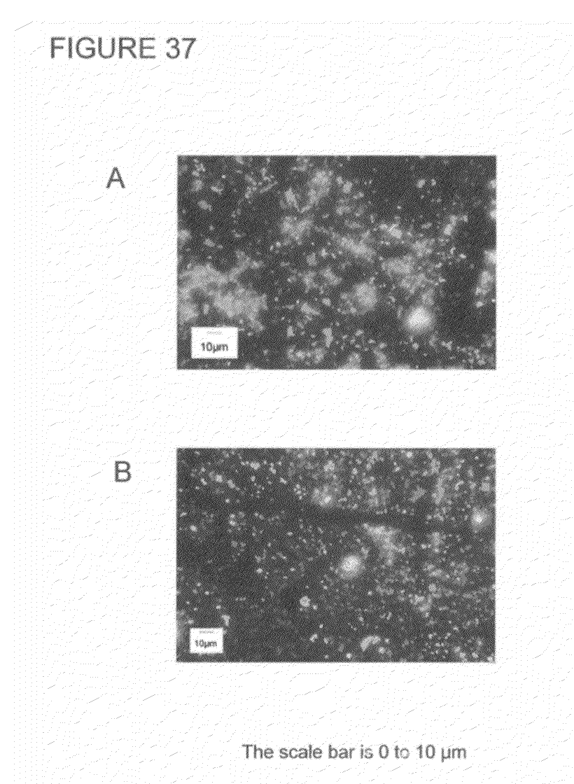

FIG. 37 shows the photomicrograph image of the non-autoclaved sample (panel A) and the autoclaved sample (Panel B). The endospores in panel A appeared brighter in green intensity than in panel B. As before the green spots are indicated by green spots (which in the fluorescence microscope image appeared as intense green spots). The results thus show that after 15 minutes of autoclaving the fluorescence intensity of the endospores has decreased, visible to the eye under the microscope. Therefore a luminescent endospore becomes less luminescence when subjected to a sterilization process. The data also indicate that there is a higher density of luminescent spores in Panel A than Panel B which means some of the endospores which are killed may have lost luminescence almost completely.

The contrast between killed and viable spores can be increased by adding a quenching metal ion (such as cobalt) to the solution prior to, during, or after, autoclaving. The quenching metal ion can penetrate non-viable spores that have the spore coat disrupted. The quenching metal ions will seek out dye molecules that have been released by the Europium ions (or other metal ions), and quench them.

Although this example has been carried out with calcein and Europium, the presently disclosed and claimed inventive concept(s) will also work with other dyes and metal ions as described herein.

In summary, the methods of the presently disclosed and claimed inventive concept(s) are applicable to near real-time detection of bacterial spores without interference biological materials such as bacteria, pollen, viruses, and fungal spores. The bacterial spores can be members of genera Bacillus (including B. anthracis), Clostridium (including C. difficile, C. botulinum, and C. perfringens) and Sporosarcina.

With reference to FIGS. 1 to 3, the presently disclosed and claimed inventive concept(s) extends to a spore 20 that has been penetrated by the reagent 5 comprising the second chelator (shown as the luminescent dye 3) and the metal ion 4. Preferably the spore 20 is luminescent. This can be achieved either by using the luminescent dye 3 (such as described with reference to FIGS. 1 to 32).

In an embodiment of the presently disclosed and claimed inventive concept(s), the metal ion 4 is a luminescent metal ion such as a lanthanide (specifically terbium or europium), and the second chelator is selected such that when bound to the luminescent metal ion 4, the second chelator is able to penetrate the spore 20. The second chelator can be selected to have a negative charge that neutralizes the positive charge of the metal ion 4. The resulting chelate is then able to penetrate the spore coat 24. Additionally or alternatively, the resulting chelate can permeate the spore coat 24 and enter into the cortex 23. Additionally or alternatively, the resulting chelate can enter into the core 21. The second chelator can be selected such that the DPA 1 within the spore 20 displaces the luminescent metal ion 4 and forms a luminescent chelate which can be excited (typically using ultra violet light) as described in reference to FIG. 30. The luminescent chelate can be Terbium-DPA or Europium-DPA. The second chelator is preferably a luminescent dye (as described with reference to FIGS. 1 to 32), but need not be. However, as the result described with reference to FIG. 30 demonstrates, a good choice for the second chelator is a luminescent dye such as calcein because it is found that the luminescence from calcein is more intense than the luminescence from either a terbium-DPA chelate or a europium-DPA chelate when compared on a molar basis.

In the methods, reagents, apparatus, and spores of the presently disclosed and claimed inventive concept(s), as further described with reference to FIGS. 1 to 30, the luminescent dye 3 can be selected to have a higher luminescence intensity than a terbium-DPA chelate when equal molar concentrations of the luminescent dye 3 and the terbium-DPA chelate are excited at their respective excitations wavelengths (λex) 82.

The spore 20 can be obtainable by a method described herein.

With reference to FIGS. 1 and 3, the presently disclosed and claimed inventive concept(s) extends to an endospore 20 that luminesces when excited by optical radiation 8, which endospore comprises a spore coat 24, at least one compartment 21, dipicolinic acid 1, a luminescent dye 3, and a plurality of a metal ion 4, wherein the spore coat 24 surrounds the compartment 21, the compartment 21 contains the dipicolinic acid 1, the luminescent dye 3 has a characteristic luminescence 7 when excited by the optical radiation 8, the endospore 20 being characterized in that: the metal ions 4 when bound to the luminescent dye 3 reduce the luminescence 7 of the luminescent dye 3 and increase the rate of diffusion of the luminescent dye 3 through the spore coat 24; the metal ions 4 bind to the dipicolinic acid 1 in preference to binding to the luminescent dye 3; and thus when the luminescent dye 3 is bound to the metal ion 4 and diffuses through the spore coat 24, it reacts with the dipicolinic acid 1, releasing the metal ion 4 to the dipicolinic acid 1, increases the luminescence of the dye 3, and results in an endospore 20 that luminesces when excited by the optical radiation 8.

As described with reference to FIGS. 1 to 37, the bacterial endospore 20 may comprise dipicolinic acid 1 or a derivative thereof. The luminescent material 333 may be a luminescent dye 3. The luminescent dye 3 may have been introduced into the endospore 20 by binding it to a metal ion 4 to form a dye-metal complex 13 and contacting the endospore 20 with the dye-metal complex 13.

The luminescent dye 3 may be quenched by the metal ion 4.

The dye-metal complex 13 may become unquenched when the luminescent dye 3 is released from the dye-metal complex 13.

The luminescent dye 3 may be such that itself quenches above a certain concentration, and the concentration of the luminescent dye 3 in the compartment 21 to 25 may be such that the luminescent dye 3 is self quenched prior to the sterilization and/or disinfection process. The luminescent dye 3 may become unquenched as a result of the sterilization and/or disinfection process.

The luminescent dye 3 may be such that it is not quenched by calcium.

The luminescent dye 3 may be a fluorescent dye. The fluorescent dye may be selected from the group comprising the following and their derivates: Calcein, Fluorescein, rhodamine, texas red, Alexa fluor, DyLight, Cy3 and Cy5, Quantum dots and near infra red dyes, fura dyes, or resorufin.

The metal ion 4 may be selected from the group comprising a rare earth metal ion, cobalt, copper, nickel, zinc, manganese, iron, lead, cadmium and mercury.

The metal ion 4 may be a rare earth ion. The rare earth ion may be terbium or europium.

The luminescence of the dye 3 in a viable spore 20 may be greater than the luminescence of the dye in a non-viable spore.

The luminescence of the dye 3 in a viable spore 20 may be lower than the luminescence of the dye in a non-viable spore.

The method may include the step of providing a quenching chemical. The quenching chemical may be cobalt ions.

In an embodiment of the presently disclosed and claimed inventive concept(s), the live organism 332 is a fungal spore, a protozoan spore or cyst, a dehydrated animal or protozoan cell, a virus, a fungal mycelium, or a bacterial cell.

As shown in FIG. 34, the presently disclosed and claimed inventive concept(s) extends to an autoclave 340 whose sterilization has been confirmed using one of the preceding methods.

It is to be appreciated that the embodiments of the presently disclosed and claimed inventive concept(s) described above with reference to the accompanying drawings have been given by way of example only and that modifications and additional components may be provided to enhance performance. In particular, the embodiments disclosed are without limitation to any metal ions or dye mentioned specifically. The presently disclosed and claimed inventive concept(s) extends to the above-mentioned features taken in isolation or in any combination.

Thus, in accordance with the presently disclosed and claimed inventive concept(s), there has been provided a method for determining the effectiveness of a sterilization and/or disinfection process that fully satisfies the objectives and advantages set forth hereinabove. Although the presently disclosed and claimed inventive concept(s) has been described in conjunction with the specific drawings, experimentation, results and language set forth hereinabove, it is evident that many alternatives, modifications, and variations will be apparent to those skilled in the art. Accordingly, it is intended to embrace all such alternatives, modifications and variations that fall within the spirit and broad scope of the presently disclosed and claimed inventive concept(s).

1-20. (canceled)

21. A method for determining the effectiveness of a sterilization and/or disinfection process, said method comprising the steps of:
(a) exposing at least one live organism to a sterilization and/or disinfection process, wherein a luminescent material is introduced into the organism prior to or following the sterilization and/or disinfection process, and wherein the live organism comprises a permeability layer that retains the luminescent material, and wherein the at least one organism having the luminescent material introduced therein forms a biological indicator;
(b) exciting the biological indicator with an excitation energy distinctive of the luminescent material;
(c) measuring the luminescence of the biological indicator;
(d) determining the effectiveness of the sterilization and/or disinfection process based on step (c), wherein killing of the organism by the sterilization and/or disinfection process permits the luminescent material to pass through the permeability layer of the organism, thereby changing the luminescence of the biological indicator.

22. The method of claim 21, wherein the luminescent material is introduced into the organism prior to exposure to the sterilization and/or disinfection process.

23. The method of claim 21, wherein the luminescent material is introduced into the organism following exposure to the sterilization and/or disinfection process.

24. The method of claim 21, wherein the at least one live organism is in a viable and dehydrated state.

25. The method of claim 21, wherein the determination step is based on an initial known number of live organisms present in the biological indicator.

26. The method of claim 21, wherein the live organism is a bacterial endospore and the permeability layer is a spore coat.

27. The method of claim 26, wherein the bacterial endospore comprises dipicolinic acid or a derivative thereof, the luminescent material is a luminescent dye, and the luminescent dye has been introduced into the endospore by binding it to a metal ion to form a dye-metal complex and contacting the endospore with the dye-metal complex.

28. The method of claim 27, wherein the luminescent dye is quenched by the metal ion.

29. The method of claim 28, wherein the dye-metal complex becomes unquenched when the luminescent dye is released from the dye-metal complex upon contact with DPA from an endospore.

30. The method of claim 29, wherein the luminescent dye is such that it self-quenches above a certain concentration, and the concentration of the luminescent dye in the compartment is such that the luminescent dye is self-quenched prior to the sterilization and/or disinfection process.

31. The method of claim 29, wherein the luminescent dye becomes unquenched as a result of the sterilization and/or disinfection process.

32. The method of claim 27, wherein the luminescent dye is such that it is not quenched by calcium.

33. The method of claim 27, wherein the luminescent dye is a fluorescent dye selected from the group consisting of Calcein, Fluorescein, rhodamine, texas red, Alexa fluor, DyLight, Cy3 and Cy5, Quantum dots and near infra red dyes, fura dyes, resorufin, and derivatives thereof and combinations thereof.

34. The method of claim 27, wherein the metal ion is selected from the group comprising a rare earth metal ion, cobalt, copper, nickel, zinc, manganese, iron, lead, cadmium and mercury.

35. The method of claim 34, wherein the rare earth ion is selected from the group consisting of lanthanum, cerium, praseodymium, neodymium, promethium, samarium, europium, gadolinium, terbium, dysprosium, holmium, erbium, thulium, ytterbium, lutetium, and combinations thereof.

36. The method of claim 27, wherein the luminescence of the dye in a viable spore is greater than the luminescence of the dye in a non-viable spore.

37. The method of claim 27, wherein the luminescence of the dye in a viable spore is lower than the luminescence of the dye in a non-viable spore.

38. The method of claim 21, further comprising the step of adding a quenching chemical to reduce background and non-specific signal arising from free dye.

39. The method of claim 38, wherein the quenching chemical comprises cobalt ions.

40. A method of claim 21, wherein the live organism is at least one of a fungal spore, a protozoan spore or cyst, a dehydrated animal or protozoan cell, a virus, a fungal mycelium, and a bacterial cell.

41. An autoclave in which a sterilization process is performed, wherein an effectiveness of a sterilization process performed therein is determined by the method of claim 21.
